(19) United States
(12) Patent Application Publication
(10) Pub. No.: US 2026/0308700 A1
(43) Pub. Date: Oct. 8, 2026

(54) Systems and Methods For The Identification And Characterization Of Pathogens In Urine Samples To Inform Patient Treatment

(71) Applicant: Biotia, Inc., Tappan, NY (US)

(72) Inventors: Niamh B. O'Hara, Tappan, NY (US); Mara Couto-Rodriguez, Brooklyn, NY (US); Dorottya Nagy-Szakal, Brooklyn, NY (US); David C. Danko, Brooklyn, NY (US)

(21) Appl. No.: 19/635,969

(22) Filed: Apr. 1, 2026

Related U.S. Application Data
(60) Provisional application No. 63/783,550, filed on Apr. 4, 2025.

(51) Int. Cl. C12Q 1/689; C12N 15/10; C12Q 1/6895; G16B 15/10; G16B 20/20; G16B 30/00; G16B 40/00

(52) U.S. Cl. CPC C12Q 1/689; C12N 15/1093; C12Q 1/6895; G16B 15/10; G16B 20/20; G16B 30/00; G16B 40/00; C12Q 2600/156

(57) ABSTRACT

The disclosure describes a system combining a clinical-grade next-generation sequencing (NGS)-based diagnostic assay and software systems for the detection, identification, and characterization of pathogens in urine specimens to inform treatment of urogenital infections. The system is trained and incorporates a bioinformatic pipeline that uses machine learning (ML) to identify pathogens based on the NGS output from the urine sample.

### CROSS-REFERENCE TO RELATED APPLICATIONS

This application claims priority to U.S. Provisional Patent Application No. 63/783,550, filed Apr. 4, 2025, which is incorporated by reference in its entirety.

### TECHNICAL FIELD

The present disclosure relates to optimized nucleic acid extraction and machine learning analysis, specifically focusing on a genomic and machine learning-based assay for pathogen identification and characterization in clinical specimens. This method aims to inform treatment of patients with infections, thereby improving health outcomes.

### SUMMARY

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

In the realm of clinical diagnostics, the accurate and efficient extraction of nucleic acids from clinical specimens is crucial for the identification and analysis of pathogens. Traditional methods often require large sample volumes and can be hindered by the presence of inhibitors that affect the accuracy of downstream analyses. This is particularly challenging in the context of urine samples, which are commonly used for diagnosing a variety of infections but present unique difficulties due to their composition and the presence of inhibitory substances.

Moreover, the rise of antimicrobial resistance and the need for precision medicine have underscored the importance of developing advanced diagnostic tools that can not only identify pathogens but also provide insights into their virulence and resistance profiles. Machine learning and genomic technologies have emerged as powerful tools in this regard, offering the potential to enhance the speed and accuracy of pathogen detection and characterization. However, integrating these technologies into routine clinical practice requires robust protocols that can handle the complexities of clinical specimens while providing comprehensive pathogen coverage. The development of such protocols is essential to improve diagnostic outcomes and guide effective treatment strategies, particularly for infections that are difficult to diagnose and treat, such as urinary tract infections and bacterial vaginosis.

By way of background, conventional infectious disease diagnostics (such as culture) are limited, slow and, 30-60% of tests fail to provide a diagnosis. This problem is even more evident for hard-to-culture pathogens (e.g. gram-negative bacteria, fungi, and less common pathogens). Furthermore, antimicrobial resistance (AMR) is a leading cause of death around the world (4.95 million AMR-related death in 2019), and annually 10 million AMR-associated deaths are predicted worldwide by 2050. Infections of concern range from recurrent and complicated UTIs, to bacterial vaginosis, to prosthetic joint infections, and other hard to diagnose infections.

Precise pathogen detection and characterization is required to inform and improve patient care, and fight drug resistance. Using a genomic and machine learning (ML)-based approach, the causative pathogen can be accurately identified along with virulence factors and antimicrobial resistance to guide precision infectious disease treatment. Computational advances (e.g. processing genomic data quickly and improved machine learning approaches) have improved turnaround time and accuracy, making this technology now useful in healthcare.

The inventive concepts discussed herein describe an optimized genomics and machine learning-based assay to identify and characterize pathogens, including antimicrobial resistance and virulence factors in clinical specimens. Different types of clinical specimens require different lab workflows for handling the sample before sequencing as well as analysis of the data post sequencing, as well as development of coupled software pipelines and training of algorithms. While certain sample types are more challenging, including urine which has low yield and higher levels of inhibitors, such issues were addressed through novel inventive concepts disclosed herein. In some aspects inhibitors, including salt crystals commonly present in urine and others, are removed from the urine sample before preparation of the nucleic acids. In other instances, the target cells have, e.g., cell walls or other harder exterior surfaces that render them difficult to access. Additional steps designed to rupture those cells and release their nucleic acids has improved the workflow of methods of the invention. Additionally computational tools available are often generalized and suffer from high false positive rates, which have been addressed in the present application through novel software pipelines and ML algorithms.

In some aspects, the disclosure describes a urine assay, comprising the steps of: extracting a set of genomic nucleic acids from a urine sample of a subject, whereby the extracting comprises the steps of: removing one or more inhibitors from the urine sample; submitting one or more microorganisms in the urine sample to one or more cycles of heating and cooling thus releasing a subset of genomic nucleic acids from the urine sample; converting the subset of genomic nucleic acids into a library of fragments that can be sequenced on a next-generation sequencing instrument; sequencing the extracted genomic DNA in the next-generation metagenomics sequencing instrument thereby generating a plurality of sequencing reads; uploading the plurality of sequencing reads into a system comprising: a memory comprising computer readable instructions stored thereon; and a processor configured to execute the computer readable instructions to: receive the plurality of sequencing reads; determine whether the plurality of sequencing reads meet one or more thresholds in a pangenome index or a duplication index; and outputting a result of the assay. In some aspects, the result of the assay is an identification of one or more antibiotic resistance markers in the sample. In some aspects, the result of the assay is an identification of bacterial vaginosis, in such instances the assay may further provide a Nugent score in the identification of bacterial vaginosis. In some instances, the pangenome index integrates sequences from the plurality of sequencing reads into a unified structure, and calculates its index as the number of genomes in which the plurality of sequencing reads are found, divided by the total number of genomes. In some instances, the duplication index is calculated as the number of pangenomes in which gene duplications are found, divided by the total number of pangenomes in the taxonomic group and expressed as a percentage. In some instances, the one or more thresholds comprise the pangenome index and the duplication index. In some instances, the incorporation of the pangenome index increases a sensitivity of the assay to greater than 85% percent. In some instances, the incorporation of the duplication index increases a specificity of the assay to greater than 85% percent. In some instances, the pangenome index and the duplication index identify greater than 70 distinct pangenomes associated with at least 70 distinct microorganisms. In some instances, the assay simultaneously screens the urine sample for at least 78 distinct microorganisms. In some instances, the one or more thresholds includes a tertiary fold index. In some instances, the tertiary fold index is a prediction of a tertiary fold from a set of sequencing reads in the plurality of sequencing reads.

In some aspects, the disclosure describes a system for organizing and representing genetic diversity, comprising: a memory comprising computer readable instructions stored thereon; and a processor configured to execute the computer readable instructions to: receive a genome sequence from a reference database; determine whether the genome sequence meets a threshold; if the genome sequence does not meet the threshold, select another genome sequence from the reference database; if the genome sequence meets the threshold, process the genome sequence according to a pangenome index and a duplication index, wherein the pangenome index integrates sequences from diverse individuals into a unified structure, facilitating rapid searches and analysis of genetic variations, and the duplication index quantifies gene duplications, offering insights into evolutionary and functional consequences; generate a score for the processed genome sequence based on input from a human expert, wherein the score comprises metrics, percentages, thresholds, or ranges; transmit the score to the reference database for storage, contributing to the refinement of the database; and continuously update and refine the threshold based on classifications generated in the reference database, wherein the continuous updating is provided by the human expert via a device such that the threshold is updated in real-time. In some instances, the genetic variations include single nucleotide polymorphisms, insertions, deletions, and structural variations. In some instances, the duplication index reflects the frequency and distribution of duplicated sequences. In some instances, the processor is further configured to execute the computer readable instructions to: generate a coarse classification database using thresholds determined by the human expert to filter results, wherein the coarse classification database organizes data into broader, more general categories compared to fine-grained classifications, grouping similar items or concepts into higher-level classes, simplifying complex datasets, and reducing dimensionality; iteratively refine the thresholds based on data; build a fine classification database using the coarse classification database, wherein the fine classification database organizes biological data into highly specific and detailed categories, allowing for precise differentiation between closely related entities such as genes, proteins, or organisms, enabling researchers to identify subtle differences and make granular distinctions; determine a set of metrics by the human expert to evaluate an alignment, wherein some individual metrics are more novel than others; iteratively refine the choice of metrics based on data; and generate the score using the machine learning algorithm trained on the data with known classifications. In some instances, the score is used to update a results summary section of a report, and the report is structured to include both human-readable formats and machine-readable formats accessible via an Application Programming Interface (API).

In some aspects, the disclosure describes a computer-implemented method for organizing and representing genetic diversity, comprising: receiving a genome sequence from a reference database; determining whether the genome sequence meets a threshold; if the genome sequence does not meet the threshold, selecting another genome sequence from the reference database; if the genome sequence meets the threshold, processing the genome sequence with novel metrics, including a pangenome index and a duplication index, wherein the pangenome index integrates sequences from diverse individuals into a unified structure, facilitating rapid searches and analysis of genetic variations, and the duplication index quantifies gene duplications, offering insights into evolutionary and functional consequences; generating a score for the processed genome sequence based on input from a human expert, wherein the score comprises metrics, percentages, thresholds, or ranges; transmitting the score to the reference database for storage, contributing to the refinement of the database; and continuously updating and refining the threshold based on classifications generated in the reference database, wherein the continuous updating is provided by the human expert via a device such that the threshold is updated in real-time. In some aspects, the genetic variations include single nucleotide polymorphisms, insertions, deletions, and structural variations. In some aspects, the duplication index reflects the frequency and distribution of duplicated sequences.

In some aspects, the computer-implemented method further comprises generating a coarse classification database using thresholds determined by the human expert to filter results, wherein the coarse classification database organizes data into broader, more general categories compared to fine-grained classifications, grouping similar items or concepts into higher-level classes, simplifying complex datasets, and reducing dimensionality; iteratively refining the thresholds based on data; building a fine classification database using the coarse classification database, wherein the fine classification database organizes biological data into highly specific and detailed categories, allowing for precise differentiation between closely related entities such as genes, proteins, or organisms, enabling researchers to identify subtle differences and make granular distinctions; determining a set of metrics by the human expert to evaluate an alignment, wherein some individual metrics are more novel than others; iteratively refining the choice of metrics based on data; and generating the score using the machine learning algorithm trained on the data with known classifications. In some aspects, the score is used to update a results summary section of a report, and the report is structured to include both human-readable formats and machine-readable formats accessible via an Application Programming Interface (API).

These aspects and other features and advantages of the invention are described below in more detail.

### BRIEF DESCRIPTION OF THE DRAWINGS

The foregoing and other features and advantages of the present invention will be more fully understood from the following detailed description of illustrative embodiments taken in conjunction with the accompanying drawings in which:

FIG. 1 is a diagram describing various modules of a system of the disclosure applied in an assay of urine samples. The diagram outlines a sequence of events with information from module 1 (sample accessing, storage, and preparation), module 2 (genomic DNA extraction and quality control (QC)), module 3 (library preparation for nucleic acid sequencing), module 4 (sequencing), and module 5 (machine learning and software).

FIG. 2 is a diagram describing various steps in utilizing a method of the disclosure. FIG. 2 illustrates a user (ordering physician) creating an order on a clinical portal and sending the sample(s) to a Lab. The schematic then depicts the sequencing and uploading of the sequencing files (FASTQ) to a module of a system of the disclosure. The diagram further depicts that upon ordering, a corresponding set of entries is a module of the system comprising a Laboratory Information Management System (LIMS) database and the aforementioned FASTQ files be linked to the sample records in the LIMS database. Subsequently, a module of the disclosure, in this case the BIOTIA-DX module, processes the samples. A PDF report is generated from the result which is uploaded to the clinical portal, and which may be accessed by the user who uploaded their data.

FIG. 3A shows a reference database system with genome sequences and scoring indices connected via a network.

FIG. 3B shows a network of reference databases connected for distributed genomic data storage and access.

FIG. 4 is a flowchart depicting a method for processing genome sequences, involving threshold checks, processing with novel metrics, score generation, and continuous threshold refinement.

FIG. 5 is a flowchart showing the process of generating and processing classification databases using a machine learning algorithm, including steps for report updating and approval.

FIG. 6 is a flowchart showing the process of generating and processing a classification database for antimicrobial resistance genes using a machine learning algorithm, followed by report updates and approvals.

FIG. 7 shows a flowchart of a relearning algorithm involving training, reinforced learning, and autonomous learning steps.

FIG. 8 shows a system architecture with a CPU, memory, storage, and machine learning algorithm connected to reference databases via a network.

FIG. 9 shows a report format for patient information, results summary, and key pathogens.

FIGS. 10A-D are charts depicting an application of the BIOTIA-DX assay in next-generation sequencing drug resistance profile determinations in clinical specimens with urinary tract infections.

FIGS. 11A-C are charts depicting an application of the urine assay of the disclosure in detecting antimicrobial resistant gene markers.

FIGS. 12A-F are charts depicting an application of the urine assay of the disclosure in detecting bacterial vaginosis associated organism in urine.

It should be understood that the drawings and pictures are not necessarily to scale.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### Terminology

As used herein, “about” and the term “approximately,” means the recited quantity exactly and small variations within a limited range encompassing plus or minus 10% of the recited quantity. In other words, the limited range encompassed can include ±10%, ±9%, ±8%, ±7%, ±6%, ±5%, ±4%, ±3%, ±2%, ±1%, ±0.5%, ±0.2%, ±0.1%, ±0.05%, or smaller, as well as the recited value itself Thus, by way of example, “about 10” should be understood to mean “10” and a range no larger than “9-11”. For clarity, as used herein, designation of a range of values includes all integers within or defining the range, and all subranges defined by integers within the range.

As used herein, the term “and/or” refers to and encompasses any and all possible combinations of one or more of the associated listed items, as well as the lack of combinations when interpreted in the alternative (“or”).

As used herein, the term “or” refers to any one member of a particular list and also includes any combination of members of that list.

The singular forms of the articles “a,” “an,” and “the” include plural references unless the context clearly dictates otherwise. For example, the term “a pest” or “at least one pest” can include a plurality of pests, including mixtures thereof.

As used herein, the term “comprising” is intended to mean that the compositions and methods include the recited elements, but not excluding others. “Consisting essentially of” when used to define compositions and methods, shall mean excluding other elements of any essential significance to the composition or method. “Consisting of” shall mean excluding more than trace elements of other ingredients for claimed compositions and substantial method steps. Examples and implementations defined by each of these transition terms are within the scope of this disclosure. Accordingly, it is intended that the methods and compositions can include additional steps and components (comprising) or alternatively including steps and compositions of no significance (consisting essentially of) or alternatively, intending only the stated method steps or compositions (consisting of).

As used herein, the term “genomic locus” or “locus” (plural loci) is the specific location of a gene or DNA sequence on a chromosome. A “gene” refers to stretches of DNA or RNA that encode a polypeptide or an RNA chain that has functional role to play in an organism and hence is the molecular unit of heredity in living organisms. For the purpose of this invention it may be considered that genes include regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

As used herein, “expression of a genomic locus” or “gene expression” is the process by which information from a gene is used in the synthesis of a functional gene product. The products of gene expression are often proteins, but in non-protein coding genes such as rRNA genes or tRNA genes, the product is functional RNA. The process of gene expression is used by all known life—eukaryotes (including multicellular organisms), prokaryotes (bacteria and archaea) and viruses to generate functional products to survive. As used herein “expression” of a gene or nucleic acid encompasses not only cellular gene expression, but also the transcription and translation of nucleic acid(s) in cloning systems and in any other context. As used herein, “expression” also refers to the process by which a polynucleotide is transcribed from a DNA template (such as into an mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

The terms “polypeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component.

As used herein the term “amino acid” includes natural and/or unnatural or synthetic amino acids, including glycine and both the D or L optical isomers, and amino acid analogs and peptidomimetics.

As used herein the term “sample”, generally refers to a urine sample.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation. For example, where the disclosure describes a “pangenome index” without a “duplication index”, and vice-versa, this is also intended to provide antecedent basis for a negative limitation.

“Sequence identity” or “identity” in the context of two polynucleotides sequences makes reference to the residues in the two sequences that are the same when aligned for maximum correspondence over a specified comparison window.

“Percentage of sequence identity” includes the value determined by comparing two optimally aligned sequences (greatest number of perfectly matched residues) over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base occurs in both sequences to yield the number of matched positions, dividing the number of matched positions by the total number of positions in the window of comparison, and multiplying the result by 100 to yield the percentage of sequence identity. Unless otherwise specified (e.g., the shorter sequence includes a linked heterologous sequence), the comparison window is the full length of the shorter of the two sequences being compared.

Unless otherwise stated, the term “homology” pertains to comparative studies. Homology indicates an ancient common origin and temporal evolution and refers to structural characteristics. In comparative anatomy, it is used to compare structures in different animal species. In comparative protein biochemistry, “homology” retains the original meaning of “having a common evolutionary origin” and is used to evolutionarily define two or more proteins by locating common structural characteristics and common spatial distribution of, for instance, beta strands, helices, and folds. Accordingly, homologous protein structures are defined by spatial analyses. Measuring structural homology involves computing the geometric-topological features of a space. One approach used to generate and analyze three-dimensional (3D) protein structures is homology modeling (also called comparative modeling or knowledge-based modeling). Homology modeling works by finding similar sequences on the basis of the obvious fact that 3D similarity reflects 2D similarity. Nonetheless, it is important to note that homologous structures do not imply sequence similarity as a necessary condition. Sequence identity is the amount of characters which match exactly between two different sequences. Hereby, gaps are not counted and the measurement is relational to the shorter of the two sequences. This has the effect that sequence identity is not transitive, i.e. if sequence A=B and B=C then A is not necessarily equal C (in terms of the identity distance measure): A: AAGGCT, B: AAGGC, C: AAGGCAT/Here identity(A,B)=100% (5 identical nucleotides/min (length(A),length(B))). Identity(B,C)=100%, but identity(A,C)=85% ((6 identical nucleotides/7)). So 100% identity does not mean two sequences are the same. Sequence similarity is first of all a general description of a relationship but nevertheless its more or less common practice to define similarity as an optimal matching problem (for sequence alignments or unless defined otherwise). Hereby, the optimal matching algorithm finds the minimal number of edit operations (inserts, deletes, and substitutions) in order to transform the one sequence into an exact copy of the other sequence being aligned (edit distance). Using this, the percentage sequence similarity of the examples above are sim(A,B)=60%, sim(B,C)=60%, sim(A,C)=86% (semi-global, sim=1-(edit distance/unaligned length of the shorter sequence)). But there are other ways to define similarity between two objects (e.g. using tertiary structure of proteins).

Statistically significant means p≤0.05.

### DETAILED DESCRIPTION

### I. Overview

All of the functionalities described in connection with one embodiment of the methods, compositions, or formulations described herein are intended to be applicable to the additional embodiments of the methods, compositions, or formulations described herein except where expressly stated or where the feature or function is incompatible with the additional embodiments. For example, where a given feature or function of component is expressly described in connection with one embodiment but not expressly mentioned in connection with an alternative embodiment, it should be understood that the feature or component may be deployed, utilized, or implemented in connection with the alternative embodiment unless the feature or component is incompatible with the alternative embodiment.

The inventive concepts disclosed herein are directed to developing and optimizing a robust strategy on pooling clinical specimens to a multiplex assay with sufficient genome coverage yield to all urogenital pathogens including bacteria, fungi, parasites, and DNA viruses. According to the inventive concepts disclosed herein, for optimized nucleic acid extraction with minimal clinical specimen, a robust and precise protocol for sample processing is established which includes lab steps coupled with machine learning analysis. These steps include—(i) using about 2 milliliter (mL) of urine, pelleting the cells through centrifugation, and resuspending in a buffer to help remove inhibitors; (ii) removal of inhibitor(s) by using Qiagen's PowerSoil® DNA Isolation kit and buffer from MolBio. As one would appreciate, the Qiagen PowerSoil® DNA Isolation Kit is used for the extraction of high-quality DNA from a variety of soil samples. It is particularly effective at isolating DNA from complex soil types that contain humic acids and other inhibitors that can interfere with downstream applications such as PCR. The kit's bead-beating technology and optimized buffer system efficiently lyse cells and remove contaminants, yielding purified DNA suitable for microbial community analysis and other molecular biology applications. The kit is designed to remove PCR inhibitors and recover both Gram-positive and Gram-negative bacterial DNA.

Generally, urine assays have trouble identifying fungi and gram-positive bacteria. To circumvent such issues, inventive concepts include centrifugation, bead beating, and heating and cooling steps to break open cells to capture pathogens without losing important species. Further, signal of microbial reads can be amplified to human reads by using the human genome as well as genomes from other apes for mapping human reads for removal from downstream analysis.

The inventive concepts disclosed herein may establish a rigorous protocol of using three external controls and one internal positive control for standardization and clinical-grade results. In particular, (i) Positive Control (PC)—an external control containing two yeasts, three Gram negative, and five Gram positive bacteria for validating reagent integrity and assay performance; (ii) Negative Extraction Control (NEC)—a negative urine matrix is used to evaluate extraction reagent performance and cross contamination; and (iii) No Template Control (NTC)—a negative control for monitoring reagent purity and library preparation cross contamination. Further, Internal Positive Control (IPC) may be applied, which is a spike in control (Halobacterium salinarum) that is used to assess clinical specimen integrity and performance to minimize false negative results due to inhibition. The species used is a member of the domain Archaea and known as an extremophile due to the ability to survive in high salt concentration (cannot be found in clinical specimens). Other extremophiles have been used in the oncology field for human genome studies; however, it has not been utilized in clinical infectious disease diagnostics.

There are a number of different applications for the assay disclosed herein as to diagnosing and guiding treatment of different infections including urinary tract infections (UTIs), especially recurrent or complicated UTIs. It also has clinical applications for sexually transmitted infections (STIs), prosthetic joint infections, challenging infections, and women's health. In the field of women's health, inventive concepts disclosed herein have developed an assay to use genomic data generated in the lab component of the assay in order to train machine learning algorithm to assess and diagnose bacterial vaginosis (BV) from urine by developing an algorithm of an (NGS)-based Nugent score based on compositional dynamics of lactobacilli and BV-associated organisms. As a skilled artisan would appreciate, an (NGS)-based Nugent score uses next-generation sequencing (NGS) to analyze vaginal microbiota and assign a score, similar to the traditional Nugent score, to assess the presence and types of bacteria associated with bacterial vaginosis (BV). This has been incorporated into the assay of the inventive concepts disclosed herein to improve diagnostic capabilities, and inform selection of antimicrobials to treat patients and improve outcomes.

The laboratory assay disclosed herein supports improved pathogen detection and related antimicrobial resistance (AMR) gene characterization and prediction of antimicrobial resistance from clinical specimens. The distinction of true positive species and resistance versus background, commensals, low false positive species can help to improve patient diagnostics, and targeted therapeutics. The inventive concepts disclosed herein include lab and analysis tools as part of the product to differentiate between background contamination and an acute infection that warrants treatment in order to inform treatment options and improve patient outcomes. These include calibrating the genomics-based assay using clinical culture cutoffs, building databases, and analytics to analyze a pathogen in a sample and characterize its genome including its virulence and drug resistance genes to characterize it as a more or less pathogenic strain in order to inform treatment.

The assay, including the software components disclosed, can be used to identify pathogens and resistance markers from not only urine but other clinical specimens including but not limited to sterile fluids (joint fluid, cerebrospinal fluid, pleural fluid, peritoneal fluid, etc.), blood and blood components (serum, plasma), tissue and biopsy specimens, respiratory specimens, clinical swabs, stool and discharges.

In some aspects, the disclosure describes a urine assay, comprising the steps of: extracting a set of genomic nucleic acids from a urine sample of a subject, whereby the extracting comprises the steps of: removing one or more inhibitors from the urine sample; submitting one or more microorganisms in the urine sample to one or more cycles of heating and cooling thus releasing a subset of genomic nucleic acids from the urine sample; converting the subset of genomic nucleic acids into a library of fragments that can be sequenced on a next-generation sequencing instrument; sequencing the extracted genomic DNA in the next-generation metagenomics sequencing instrument thereby generating a plurality of sequencing reads; uploading the plurality of sequencing reads into a system comprising: a memory comprising computer readable instructions stored thereon; and a processor configured to execute the computer readable instructions to: receive the plurality of sequencing reads; determine whether the plurality of sequencing reads meet one or more thresholds in a pangenome index or a duplication index; and outputting a result of the assay. In some aspects, the result of the assay is an identification of one or more antibiotic resistance markers in the sample. In some aspects, the result of the assay is an identification of bacterial vaginosis, in such instances the assay may further provide a Nugent score in the identification of bacterial vaginosis. In some instances, the pangenome index integrates sequences from the plurality of sequencing reads into a unified structure, and calculates its index as the number of genomes in which the plurality of sequencing reads are found, divided by the total number of genomes. In some instances, the duplication index is calculated as the number of pangenomes in which gene duplications are found, divided by the total number of pangenomes in the taxonomic group and expressed as a percentage. In some instances, the one or more thresholds comprise the pangenome index and the duplication index. In some instances, the incorporation of the pangenome index increases a sensitivity of the assay to greater than 85% percent. In some instances, the incorporation of the duplication index increases a specificity of the assay to greater than 85% percent. In some instances, the pangenome index and the duplication index identify greater than 70 distinct pangenomes associated with at least 70 distinct microorganisms. In some instances, the assay simultaneously screens the urine sample for at least 78 distinct microorganisms. In some instances, the one or more thresholds includes a tertiary fold index. In some instances, the tertiary fold index is a prediction of a tertiary fold from a set of sequencing reads in the plurality of sequencing reads.

In some aspects, the disclosure describes a system for organizing and representing genetic diversity, comprising: a memory comprising computer readable instructions stored thereon; and a processor configured to execute the computer readable instructions to: receive a genome sequence from a reference database; determine whether the genome sequence meets a threshold; if the genome sequence does not meet the threshold, select another genome sequence from the reference database; if the genome sequence meets the threshold, process the genome sequence according to a pangenome index and a duplication index, wherein the pangenome index integrates sequences from diverse individuals into a unified structure, facilitating rapid searches and analysis of genetic variations, and the duplication index quantifies gene duplications, offering insights into evolutionary and functional consequences; generate a score for the processed genome sequence based on input from a human expert, wherein the score comprises metrics, percentages, thresholds, or ranges; transmit the score to the reference database for storage, contributing to the refinement of the database; and continuously update and refine the threshold based on classifications generated in the reference database, wherein the continuous updating is provided by the human expert via a device such that the threshold is updated in real-time. In some instances, the genetic variations include single nucleotide polymorphisms, insertions, deletions, and structural variations. In some instances, the duplication index reflects the frequency and distribution of duplicated sequences. In some instances, the processor is further configured to execute the computer readable instructions to: generate a coarse classification database using thresholds determined by the human expert to filter results, wherein the coarse classification database organizes data into broader, more general categories compared to fine-grained classifications, grouping similar items or concepts into higher-level classes, simplifying complex datasets, and reducing dimensionality; iteratively refine the thresholds based on data; build a fine classification database using the coarse classification database, wherein the fine classification database organizes biological data into highly specific and detailed categories, allowing for precise differentiation between closely related entities such as genes, proteins, or organisms, enabling researchers to identify subtle differences and make granular distinctions; determine a set of metrics by the human expert to evaluate an alignment, wherein some individual metrics are more novel than others; iteratively refine the choice of metrics based on data; and generate the score using the machine learning algorithm trained on the data with known classifications. In some instances, the score is used to update a results summary section of a report, and the report is structured to include both human-readable formats and machine-readable formats accessible via an Application Programming Interface (API).

In some aspects, the disclosure describes a computer-implemented method for organizing and representing genetic diversity, comprising: receiving a genome sequence from a reference database; determining whether the genome sequence meets a threshold; if the genome sequence does not meet the threshold, selecting another genome sequence from the reference database; if the genome sequence meets the threshold, processing the genome sequence with novel metrics, including a pangenome index and a duplication index, wherein the pangenome index integrates sequences from diverse individuals into a unified structure, facilitating rapid searches and analysis of genetic variations, and the duplication index quantifies gene duplications, offering insights into evolutionary and functional consequences; generating a score for the processed genome sequence based on input from a human expert, wherein the score comprises metrics, percentages, thresholds, or ranges; transmitting the score to the reference database for storage, contributing to the refinement of the database; and continuously updating and refining the threshold based on classifications generated in the reference database, wherein the continuous updating is provided by the human expert via a device such that the threshold is updated in real-time. In some aspects, the genetic variations include single nucleotide polymorphisms, insertions, deletions, and structural variations. In some aspects, the duplication index reflects the frequency and distribution of duplicated sequences.

In some aspects, the computer-implemented method further comprises generating a coarse classification database using thresholds determined by the human expert to filter results, wherein the coarse classification database organizes data into broader, more general categories compared to fine-grained classifications, grouping similar items or concepts into higher-level classes, simplifying complex datasets, and reducing dimensionality; iteratively refining the thresholds based on data; building a fine classification database using the coarse classification database, wherein the fine classification database organizes biological data into highly specific and detailed categories, allowing for precise differentiation between closely related entities such as genes, proteins, or organisms, enabling researchers to identify subtle differences and make granular distinctions; determining a set of metrics by the human expert to evaluate an alignment, wherein some individual metrics are more novel than others; iteratively refining the choice of metrics based on data; and generating the score using the machine learning algorithm trained on the data with known classifications. In some aspects, the score is used to update a results summary section of a report, and the report is structured to include both human-readable formats and machine-readable formats accessible via an Application Programming Interface (API).

Additional details are provided in the following sections.

### II. BIOTIA-ID Urine NGS Assay

In some aspects, the invention relates to an optimized assay, e.g., BIOTIA-ID Urine NGS assay, comprising optimized processes for nucleic acid extraction from urine samples, standardized controls, a multiplex assay, and software for microbial species classification and antimicrobial resistance detection.

### Nucleic Acid Extraction Protocol, Sample Processing and Urine Inhibitor Removal

In some aspects, an assay of the disclosure comprises a process for the extraction and analysis of nucleic acids from urine samples that includes a step of removing inhibitors commonly found in urine to improve the accuracy and reliability of diagnostic assays.

In some aspects, the disclosure addresses the limitations of current diagnostics by optimizing nucleic acid extraction from urine samples. Urine samples are frequently used in clinical diagnostics due to their non-invasive collection method. However, urine samples may contain various inhibitors that can interfere with nucleic acid extraction and downstream analysis. Some inhibitors may be naturally occurring proteins, others may be non-naturally occurring drugs administered to the subject. Common exemplary inhibitors include uric acid crystals, calcium phosphate, amorphous phosphate crystals, calcium oxalate crystals, ACE inhibitors, ARBs, SGLT2 inhibitors, and inhibitory macromolecules such as crystal matrix protein, bikunin, Tamm-Horsfall protein (THP), and urinary osteopontin (OPN). These inhibitors can pose significant challenges in accurately identifying pathogens, particularly fungi and gram-positive bacteria, some directly interfere with the performance of current diagnostic assays. In some aspects, the disclosure incorporates a step of removing such inhibitors from a urine sample prior to nucleic acid analysis. In some instances, a step of removing the inhibitors can greatly improve the sensitivity and/or specificity of the assay.

### Urine Sample Collection

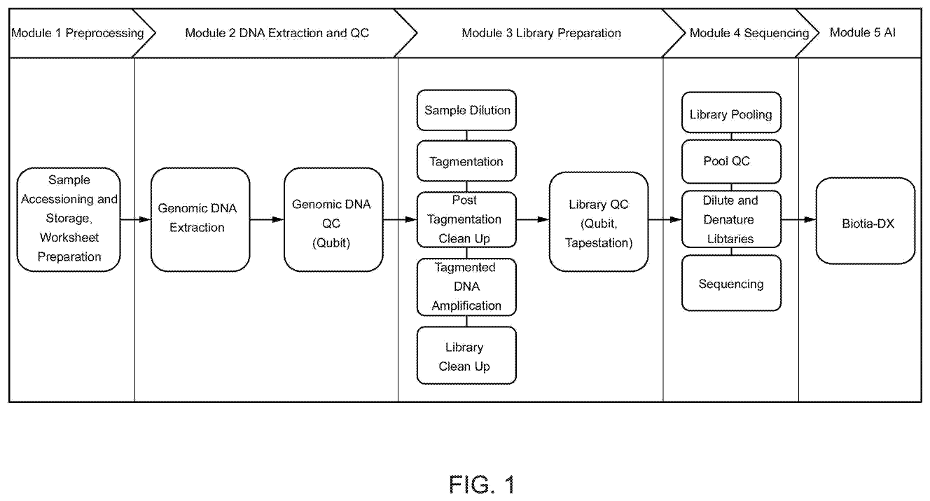

In some aspects, the disclosure describes an assay comprising a step of collecting or receiving, storing, and preparing a urine sample from a subject. See FIG. 1, Module 1, preprocessing. In many instances, an assay of the disclosure requires no more than 10 mL, no more than 9 mL, no more than 8 mL, no more than 7 mL, no more than 6 mL, no more than 5 mL, no more than 4 mL, no more than 3 mL, or no more than 2 mL, or no more than 1 mL of a urine sample. In many instances, an assay of the disclosure requires from 0.1 mL to 5 mLs, from 0.5 mL to 5 mLs, from 1 mL to 5 mLs, from 1.5 mL to 5 mLs, from 0.1 mL to 4 mLs, from 0.5 mL to 4 mLs, from 1 mL to 4 mLs, from 1.5 mL to 4 mLs, from 0.1 mL to 3 mLs, from 0.5 mL to 3 mLs, from 1 mL to 3 mLs, from 1.5 mL to 3 mLs of a urine sample. In many instances, an assay of the disclosure utilizes about 2 mLs of a urine sample.

### Urine Sample Processing: Removal of Inhibitors

In some aspects, the disclosure describes an assay comprising a step of nucleic acid extraction and quality control analysis (QC) from a urine sample. See FIG. 1, Module 2, DNA extraction and QC. In some aspects, the genomic DNA extraction comprises a step of pelleting cells, e.g., fungi cells, bacteria cells, subject cells, and/or debris, from a urine sample, for example via centrifugation. Centrifugation can be high speed centrifugation. The pellet can then be resuspended in a buffer or with a method that removes or inactivates a plurality of inhibitors from the urine sample, e.g., aluminium ammonium sulfate dodecahydrate, phenol-chloroform extraction, ion-exchange column chromatography, cation exchange columns, or CTAB based extraction. In some instances, the pellet is then added to a bead-beating tube for rapid and thorough homogenization. See FIG. 1, Module 2, DNA extraction and QC.

Urine Sample Processing: Heat and Cooling Steps for the Release of Genomic Nucleic Acid from Hard-to-Culture Pathogens

Current conventional infectious disease diagnostics, such as culture methods, are limited and slow, with 30-60% of tests failing to provide a diagnosis. This issue is particularly pronounced for hard-to-culture pathogens, including gram-negative bacteria, fungi, and less common pathogens. Precise pathogen detection and characterization are essential for effective antimicrobial selection, improved patient care, and combating drug resistance.

During the assay cell lysis can occur by mechanical and/or chemical methods. The disclosure incorporates additional heating and cooling cycles to further break open cells and release nucleic acids from difficult to capture cells, e.g., fungi cells.

In some aspects, total genomic DNA is captured on a silica membrane in a spin column format. DNA is then washed and eluted from the membrane and prepared for NGS, PCR, and other downstream applications. Such steps not only extract the DNA from the urine sample, but they remove inhibitors that could potentially interfere with the quality of the nucleic acid sequencing reaction. See FIG. 1, Module 2, DNA extraction and QC.

### Preparing the Sample for Nucleic Acid Sequencing Analysis

In some aspects, the disclosure describes urine assays and systems designed for analyzing high volume data generated by fully-automated, next-generation sequencing of nucleic acids extracted from urine samples. Preferably, the sequencing platform utilized is a next-generation sequencing platform, such as the Illumina NextSeq 550 platform. These systems are capable of performing simultaneous diagnosis and genomic surveillance of a wide array of microorganisms present in a urine sample.

### DNA Library Preparation for Next-Generation Sequencing

Most NGS platforms, such as the Illumina NextSeq 550 platform analyze DNA in uniform, bite-size pieces, created by DNA fragmentation. This process generates a ‘library’ of fragments with a narrow length distribution that is optimal for the sequencing platform. See FIG. 1, Modules 3 and 4, Library Preparation and Sequencing. The disclosure specifically contemplates embodiments where the nucleic acids extracted from the urine sample are prepared as a library for sequencing. See FIG. 1, Module 3, Library preparation. Library preparation steps typically include sample dilution, tagmentation, post tagmentation clean up, tagmented DNA amplification, library clean up. Typically, a quality control step is performed and the amount of DNA library is quantified (e.g., tapestation). More specifically, the NGS of the disclosure can comprise the following steps.

DNA Tagmentation/fragmentation: Both mechanical fragmentation (shearing) and enzymatic methods are suitable for NGS. Mechanical methods enable random shearing to produce a variety of overlapping fragments for any given region of the genome. This is ideal for de novo assembly. Enzymatic methods are relatively fast and require less investment upfront but have some ‘bias’, cleaving some sites preferentially, making de novo assembly more challenging without the variety of overlapping fragments.

DNA end-repair: DNA tagmentation/fragmenting can be followed by a step of end-repair. The fragments generated have single-stranded, ‘sticky’ ends. The next step, end-repair, fills in these sticky ends to create blunt ends, ready for adaptor ligation. See FIG. 1, Module 3, Library preparation.

Adaptors: adaptors are then bound to both the 5′ and the 3′ ends of the library fragments. Adaptors can be specific to the sequencing platform, but ultimately all serve to enable in-platform clonal amplification, i.e. adaptors compatible with the Illumina NextSeq 550 platform analyze. See FIG. 1, Module 4, Sequencing.

The adaptors are designed to bind to the sequencer-specific substrate, such as a patterned flow cell, contain sequences to enable amplification, and can have barcodes for fragment identification.

The systems and processes described herein enable complex multivariate analysis of nucleic acid sequencing data. This analysis can, for instance, categorize specimens based on the presence or absence of certain pathogens or the tertiary folding of the structure of the sequenced polypeptides. FIG. 1, Module 5, AI.

It is specifically contemplated that, in certain embodiments, the next-generation sequencing will produce a plurality of sequencing reads that can be directly associated by a system of the disclosure with a microorganism. See FIG. 1, Module 5, AI. It is also contemplated that the next generation sequencing will reveal one or more mutations in the gene product encoded by a plurality of the sequencing reads. Additionally, it is contemplated that the disclosed system can identify mutations over the length of a predicted three-dimensional fold of the predicted structure.

In some embodiments, the assay of the disclosure comprises the following steps:

Genomic DNA (gDNA) Extraction: Genomic DNA can be extracted from a suitable volume of urine, e.g., 2 mL of urine, with any suitable methods, e.g., using the QIACube-MDx system. Briefly, unpreserved urine samples can be lysed thereby freeing nucleic acids that were previously encapsulated by a membrane. Nucleic acids or proteins can then bind to a silica membrane or a purification resin (e.g., QIAGEN spin column) and washed to remove contaminants. Nucleic acids can then be eluted. Yields can be quantified using the Qubit Flex.

Normalization and Spiking: the normalized gDNA can be spiked with 5% internal positive control (IPC) and processed using a next-generation sequencing platform standard reagents, e.g., the Illumina DNA prep library preparation kit.

Quality Check: The libraries are quality checked for size and concentration using the Tapestation 4200 and Qubit Flex, respectively.

Pooling and Sequencing: The libraries are pooled in equimolar concentrations and sequenced on the Illumina NextSeq 550 platform.

In some embodiments, the assay is a urine assay, comprising the steps of: extracting genomic DNA from an unpreserved urine sample of a subject; converting the genomic DNA sample into a library of fragments that can be sequenced on a next-generation sequencing instrument; sequencing the extracted genomic DNA in the next-generation metagenomics sequencing instrument thereby generating a plurality of sequencing reads; uploading the plurality of sequencing reads into a system comprising: a memory comprising computer readable instructions stored thereon; and a processor configured to execute the computer readable instructions to: receive the plurality of sequencing reads; determine whether the plurality of sequencing reads meet one or more thresholds in a pangenome index and/or a duplication index; and outputting a result of the assay.

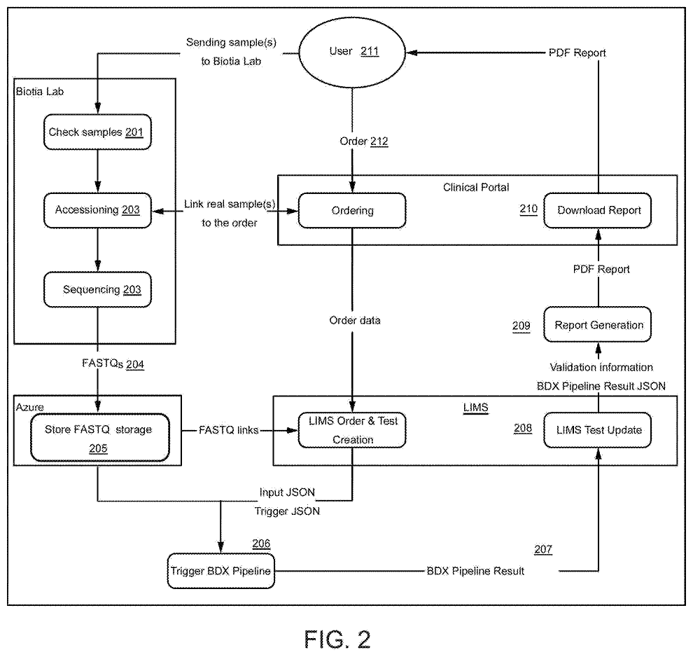

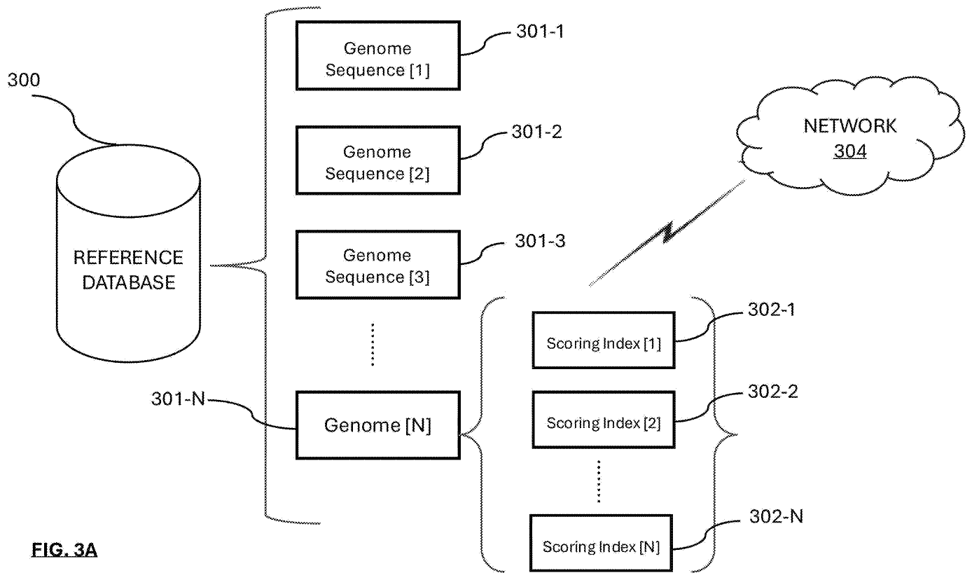

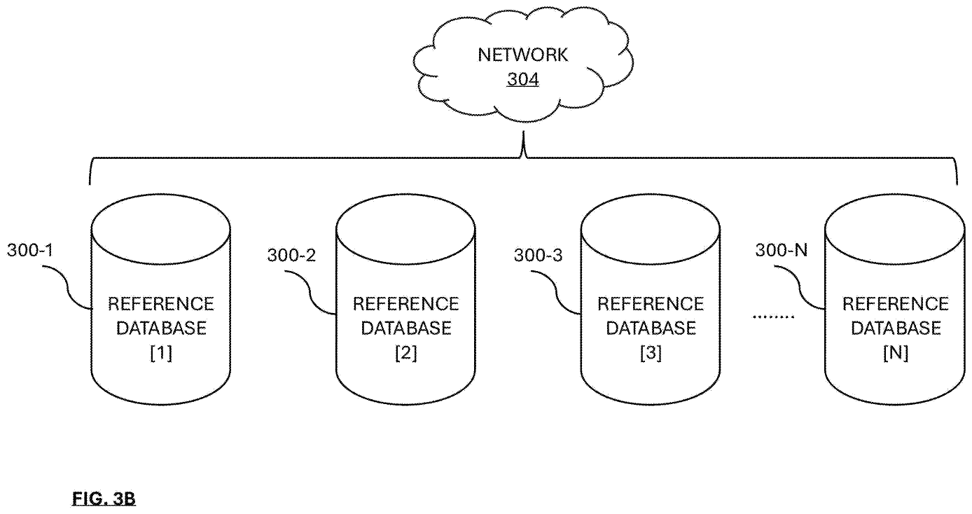

In some aspects a module of the disclosure accepts as an input a plurality of sequencing reads, e.g., in FASTQs file format or another suitable format (See FIG. 2, at 204). The files can be optionally stored (205), typically this can trigger an analysis by a software module of the disclosure (206).

### Pangenome Index

In some embodiments, the software module utilized in the pipeline analysis (206) incorporates a pangenome index. A pangenome index is an index of a group of highly similar genes calculated as the number of genomes in which these genes are found, divided by the total number of genomes. The output is expressed as a percentage (0-100%). An input for determining a pangenome index can be a plurality of nucleic acid sequencing reads determined from a set of genomes from a particular taxonomic clade (e.g. Staphylococcus aureus) where each genome consists of a set of genes that are highly similar (e.g. have >99% sequence similarity). The pangenome index of a group of highly similar genes is the number of genomes where these genes are found divided by the total number of genomes (output is a percentage 0-100%). The pangenome index of an individual gene is defined as the pangenome index of the group to which the gene belongs.

A higher pangenome index indicates that a gene is more likely to be found in a particular organism and is therefore more useful for identifying that organism, all else being equal. A module of the disclosure can output a pangenome index, predicting one set of variants from each organism as a result (207). Typically, with only one representative gene chosen from each highly similar group, e.g., one pangenome index per gene in the pangenome. Optionally, a result can be uploaded to a LIMS test platform and outputted as a report (207-209). A user can download the report (210).

### Duplication Index

In some embodiments, the software module utilized in the pipeline analysis (206) incorporates a duplication index. A set of pangenomes from a large taxonomic group (e.g., bacteria) can be used as an input for a pangenome index.

Groups of genes can be identified that exhibit sequence similarity (e.g., >95% sequence similarity). The duplication index of a group of similar genes is calculated as the number of pangenomes in which these genes are found, divided by the total number of pangenomes in the taxonomic group. The output is expressed as a percentage (0-100%). The duplication index of an individual gene is defined as the duplication index of the group to which the gene belongs. The output typically includes one duplication index per gene. A high duplication index indicates that the gene is found in many organisms within the reference set and may lead to spurious identifications. Therefore, genes with low duplication indexes are preferred.

The pangenome and duplication indices can be used to construct a reference database for taxonomic identification in a system of the disclosure. The tertiary index can be used to associate a variant with a phenotype.

In some embodiments, the assay is a urine assay, comprising the steps of: extracting genomic DNA from an unpreserved urine sample of a subject; converting the genomic DNA sample into a library of fragments that can be sequenced on a next-generation sequencing instrument; sequencing the extracted genomic DNA in the next-generation metagenomics sequencing instrument thereby generating a plurality of sequencing reads; uploading the plurality of sequencing reads into a system comprising: a memory comprising computer readable instructions stored thereon; and a processor configured to execute the computer readable instructions to: receive the plurality of sequencing reads; determine whether the plurality of sequencing reads meet one or more thresholds in a tertiary fold index; and outputting a result of the assay. See FIG. 1, Module 5, AI. The assay starts in a laboratory with sample processing (201), accessioning (202), and sequencing (203).

In some aspects a module of the disclosure accepts as an input a plurality of sequencing reads, e.g., in FASTQs file format or another suitable format (See FIG. 2, at 204). The files can be optionally stored (205), typically this can trigger an analysis by a software module of the disclosure (206).

In some embodiments, the software module utilized in the pipeline analysis (206) incorporates a tertiary fold index. Amino acid sequences are folded by biochemical processes into a three-dimensional shape referred to as a “tertiary fold.” One or more nucleic acids in the plurality of the nucleic acids extracted from the urine samples may contain variants that alter the amino acid sequence of the protein. These variants may be close to one another on the one-dimensional amino acid sequence (in which case it is more likely that they are functionally related) or they may be further apart (in which case it is less likely they are functionally related). In some aspects, variants that are far apart on the one-dimensional amino acid sequence may be close together in space in the tertiary fold. In some aspects, a module of the disclosure is designed to incorporate a tertiary fold index, a model whereby a prediction is made as to whether a nucleic acid variant in the plurality of sequencing reads is associated with a known phenotype. This prediction is made on the basis of whether a nucleic acid variant is in proximity to other variants on the “tertiary fold” even if these variants are not proximal on the amino acid sequence of the protein. The disclosure provides a module that, in some aspects, accepts nucleic acid sequences as an input and predicts from the plurality of nucleic acid sequencing reads: a) a one-dimensional amino acid sequence; and b) a tertiary fold from that amino acid sequence. A module of the disclosure can output a set of variants based on the one-dimensional amino acid sequence from multiple organisms in a population, predicting one set of variants from each organism as a result (207). Optionally, a result can be uploaded to a LIMS test platform and outputted as a report (207-209). A user can download the report (210).

In some aspects, the technique identifies spatial clusters of variants in the tertiary fold during the software analysis in the module (206). Contemplated by the disclosure are subgroups for different types of variants such as variants that convert a hydrophobic amino acid to a hydrophilic amino acid. Also contemplated are variants in these spatial clusters statistically associated with some phenotype (e.g., resistance to an antibiotic). The analysis can output the following information to a human researcher, for e.g.: 1) the location of clusters of variants (both on the one-dimensional sequence and the tertiary fold); 2) Whether these clusters are or are not associated with a particular phenotype (including subgroups of variants); 3) Information about the variant cluster on the tertiary structure such as the size of the cluster or whether it is located in a binding site or other functional region.

### III. BIOTIA-DX Software

In some aspects, the disclosure provides a computer-implemented method and system for the novel analysis of a plurality of sequence reads generated in a urine assay. In some aspects, the disclosure provides a computer-implemented method and system for organizing and representing genetic diversity through the use of a pangenome index and a duplication index. The pangenome index may integrate sequences from diverse individuals or strains into a unified structure, capturing shared and unique genomic regions, and enabling rapid searches to support analysis of genetic variation. In many instances, the data from individuals (e.g., humans) is removed from a downstream analysis, and only the non-human data is analyzed. The duplication index can quantify the extent of gene or genomic region duplications, allowing for identification and characterization of duplicated regions.

A machine learning algorithm may process genome sequences with novel metrics, generate scores, and continuously update thresholds based on classifications in a reference database. This system may include a genomic data storage and management system that stores scores locally or on a network and connects multiple reference databases, facilitating distributed data storage and access. The method and system can enhance the detection of structural variants, improve the accuracy of small variant calling, and provide insights into evolutionary and functional consequences of gene duplications. A genome sequence may be received from a reference database, and its compliance with a threshold may be determined. If the genome sequence does not meet the threshold, another genome sequence may be selected from the reference database. If the genome sequence meets the threshold, it may be processed with novel metrics, including a pangenome index and a duplication index.

The pangenome index may integrate sequences from diverse individuals or strains into a unified structure, capturing shared and unique genomic regions. The use of pangenome indexes significantly increases the sensitivity of genomic analyses compared to traditional methods. This integration may enable rapid searches across the unified structure, supporting analysis of genetic variation, which may include single nucleotide polymorphisms, insertions, deletions, and structural variations. The unified structure may provide a comprehensive representation of genetic diversity, allowing for variant detection, read alignment, and evolutionary studies. The duplication index may quantify the extent of gene or genomic region duplications, reflecting the frequency and distribution of duplicated sequences. This quantification may allow for identification and characterization of duplicated regions, providing insights into evolutionary and functional consequences. A score for the processed genome sequence may be generated using a machine learning algorithm, which may be trained on data with known classifications determined by human operators running comparator assays on samples. The score may be transmitted to the reference database for storage, and the threshold may be continuously updated and refined based on classifications generated in the reference database.

The genomic data storage and management system may include genome sequences, categorize scoring indices, store scores locally or on a network, and connect multiple reference databases, enabling data access and allowing for distributed data storage and access.

According to an embodiment, a coarse classification database may be generated using thresholds determined by a human expert, which may be iteratively refined to build a robust classification database. This process may involve determining a set of metrics to evaluate an alignment, where some metrics may be more novel than others, and iteratively refining the choice of metrics based on data. The machine learning algorithm may be trained on data with known classifications determined by human operators running comparator assays on samples. The machine learning algorithm may have human-tunable hyperparameters that can be iteratively refined by a human expert. The machine learning algorithm may process classification databases to generate a score and update a report that is transmitted to a technician and a physician.

The training of the machine learning algorithm may occur on validated data sets, enhancing learning through reinforced and autonomous learning. The machine learning algorithm may be trained to improve algorithm performance and enhance accuracy, refining classification and evaluation processes. The machine learning algorithm may be used to generate a score using the data with known classifications, which may be transmitted to the reference database for storage. The threshold may be continuously updated and refined based on classifications generated in the reference database. The machine learning algorithm may be trained to enhance learning through reinforced and autonomous learning, which may improve algorithm performance and enhance accuracy.

The duplication index may reflect the frequency and distribution of duplicated sequences, which can allow for identification and characterization of duplicated regions. This index may quantify the extent of gene or genomic region duplications, potentially assisting in clinical settings by helping to pinpoint the exact boundaries and copy numbers of duplicated genes. Such precision may be particularly useful in conditions like Charcot-Marie-Tooth disease. The duplication index may also provide insights into evolutionary and functional consequences, as it can help track gene family expansions and contractions across species. This tracking may offer insights into adaptive processes and the functional diversification of genes.

The genomic analysis may be enhanced by the increased specificity provided by the duplication index, which can allow for more precise identification and characterization of duplicated regions. The duplication index may be integrated into a broader genomic framework, where it can interact with other genomic metrics and indices to provide an analysis of genetic diversity. This integration may support applications in variant detection, read alignment, and evolutionary studies, potentially leading to a more nuanced understanding of genetic variation and its implications. A user may order a urine assay of the disclosure 211 and the ordering 212 may trigger the steps of collecting and processing the urine sample. See FIG. 2.

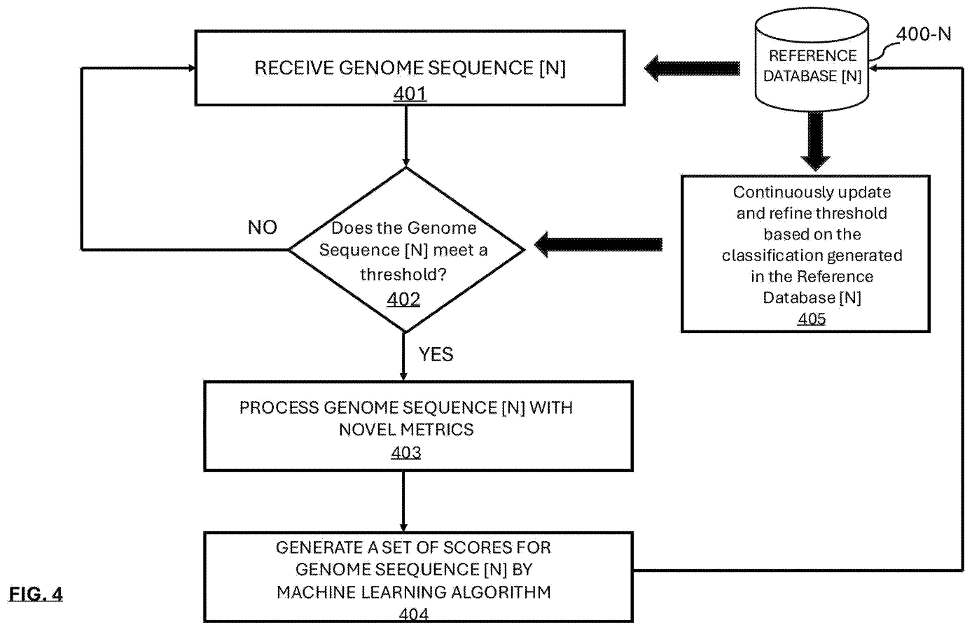

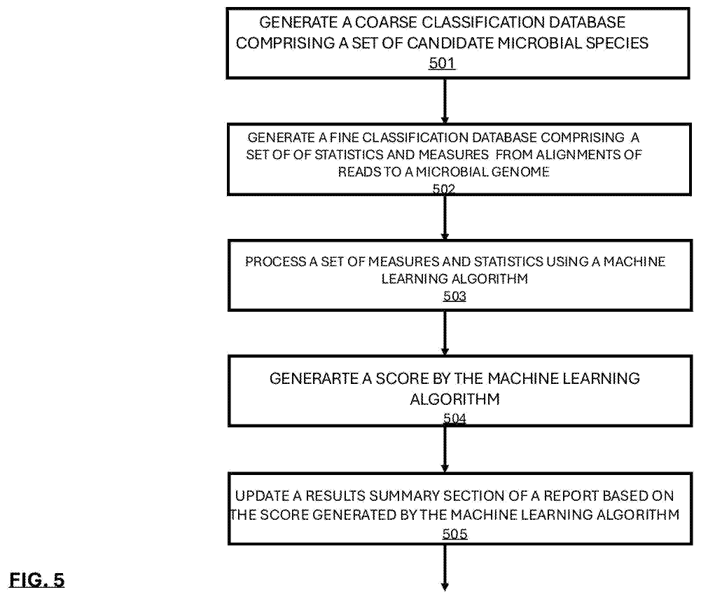

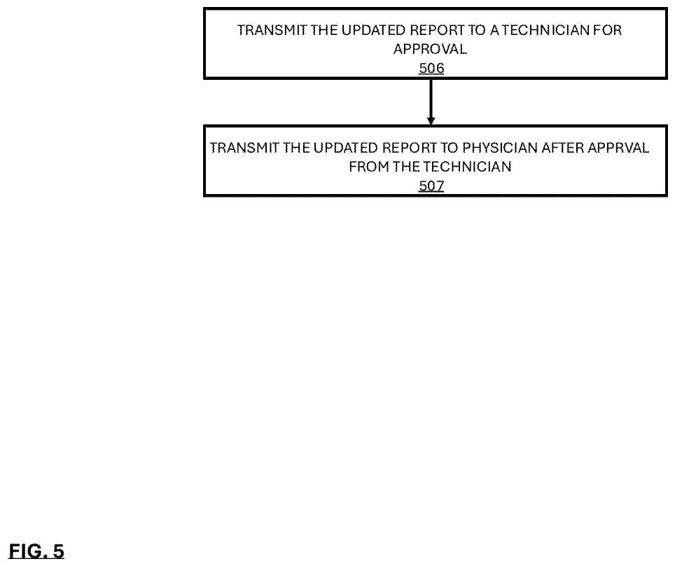

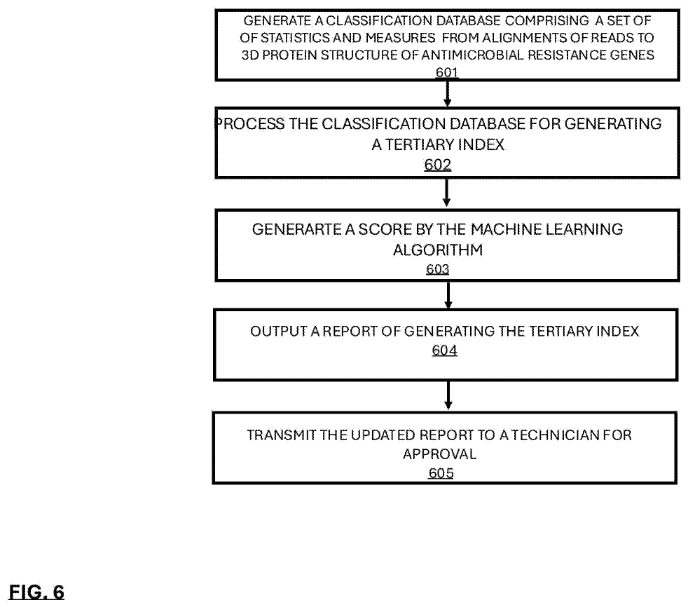

The block diagram in FIG. 3A illustrates a reference database system 300 connected to a network 304, which is integral to the genomic data storage and management system. This system is designed to organize and represent genetic diversity by categorizing genome sequences and their associated scoring indices. The reference database 300 comprises multiple genome sequences, labeled as 301-1, 301-2, 301-3, up to 301-N, where N represents a real integer. Each genome sequence is associated with a plurality of scoring indices, denoted as 302-1, 302-2, up to 302-N. These indices are used for evaluating the sequences based on their specificity and utility as genetic markers. The genome sequences within the reference database are optimized for detecting species in clinical samples, providing a framework for genomic analysis. Each scoring index is assigned a numerical value between 0 and 1, which can be as precise as the thousandth decimal point (e.g., 0.001, 0.002, 0.003, etc.). However, other values between 0 and 1 may also be assigned, for example to the tenth degree (e.g., 0.1, 0.2, 0.3, etc.), hundredth degree (e.g., 0.01, 0.02, etc.), and so forth. This scoring may be determined by human experts, and the scores are stored locally or on the network 304.

In particular, human researchers may set an initial pair of thresholds such as 50% for the pangenome index and 10% for the duplication index. However, this distribution between the pangenome index and the duplication index may be adjusted and calibrated as desired by the human researchers. In the event duplication index has a threshold set higher than 10% then such genome sequence(s) may be classified as too general or too generic. And, where the pangenome index has a threshold set lower than 50% then genome sequence(s) may be classified as too specific. Only genes or genome sequences that have a pangenome index at or above the threshold AND a duplication index at or below the threshold are included and further evaluated. And, wherein, included sequences are used to build a reference database 300 with appropriate structure and indices.

Further, validation tests are run on the reference database 300 and human researchers may inspect the results. If the results are determined to be overall too sensitive the researchers may raise the duplication index threshold or raise the pangenome index. And, if the results are overall not sensitive enough the researchers may lower the pangenome index threshold, or lower the duplication index threshold. And, lastly, if overall results are good but there are problems with particular taxa, the researchers may change the thresholds for a specific taxa according to the approach above.

Stated differently, after receiving one or more scoring indices, genome sequence is categorized into one of the following three groups: too general, too specific, or useful markers. This categorization helps in identifying the level of specificity required for genomic analysis.

The reference database system employs two key metrics: the pangenome index and the duplication index. The pangenome index integrates sequences from diverse individuals or strains into a unified structure, capturing both shared and unique genomic regions. This allows for analysis of genetic variation, including single nucleotide polymorphisms, insertions, deletions, and structural variations. Whereas, the duplication index quantifies the extent of gene or genomic region duplications, providing insights into evolutionary and functional consequences. These metrics enhance the sensitivity and specificity of genomic analyses, supporting applications in variant detection, read alignment, and evolutionary studies. The network 304 facilitates distributed data storage and access, connecting multiple reference databases. This connectivity ensures that genomic data can be managed and accessed across different platforms, enabling rapid searches and analysis of genetic diversity. The integration of these components within the reference database system underscores its capability to support genomic research and clinical applications, leveraging the power of pangenome and duplication indices to provide a detailed and accurate representation of genetic information.

FIG. 3B illustrates a networked system of reference databases connected via a network 304, forming a distributed data storage and access system. This configuration may facilitate the organization and representation of genetic diversity by integrating and managing genomic data across multiple databases. Each reference database, such as 300-1, 300-2, 300-3, and 300-N, may be part of this network, allowing for efficient data sharing and access. The network 304 may enable the system to store and categorize scoring indices, supporting the method and system for organizing genetic diversity. Each reference database, labeled as 300-1, 300-2, 300-3, and 300-N, may be identical in structure and function, containing components necessary for genomic data management. These databases may be connected to the network 304, which serves as a central hub for data exchange and communication. This system architecture may support the continuous updating and refining of thresholds based on classifications generated in the reference databases.

The integration of multiple reference databases into a networked system may enhance the ability to perform rapid searches and analyses of genetic variation, leveraging the pangenome index and duplication index. By connecting these databases 300-N, the system may provide a representation of genetic diversity, enabling applications in variant detection, read alignment, and evolutionary studies. The network 304 may facilitate the seamless flow of information, ensuring that the genomic data is up-to-date and accurately reflects the genetic diversity within a species or population. This interconnected system may be crucial for implementing the computer-implemented method and system for organizing and representing genetic diversity.

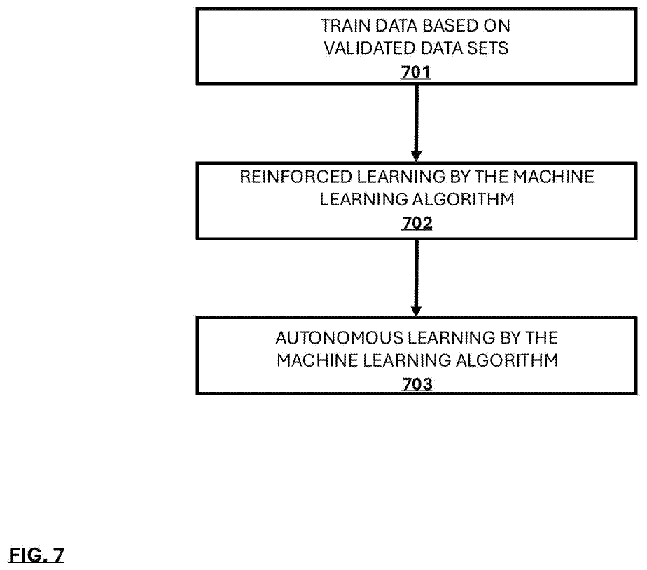

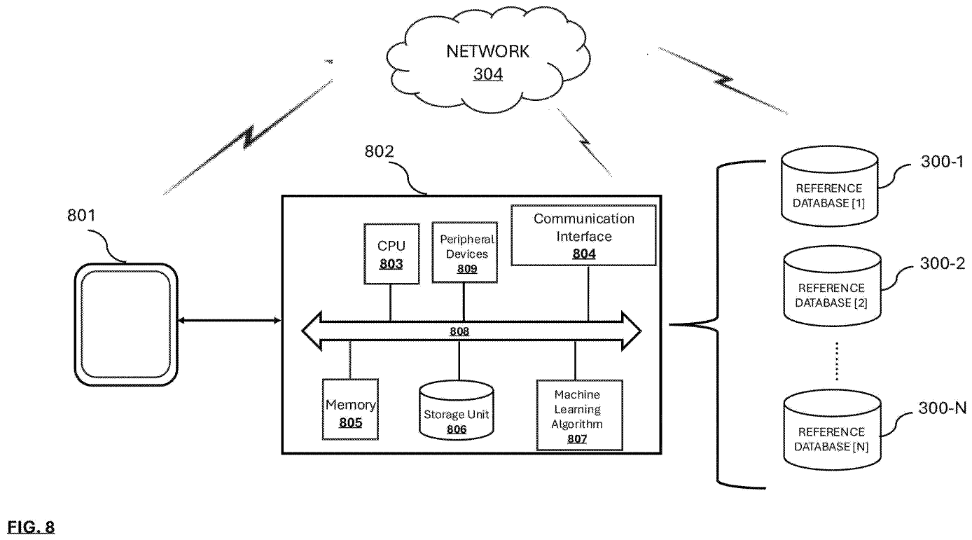

Next, FIG. 4 illustrates a flowchart depicting a computer-implemented method for organizing and representing genetic diversity, wherein the following steps of FIG. 4 may be executed one or processors configured to execute computer readable instructions. The process begins with step 401, where a genome sequence [N] is received from a reference database 300-N. This step initiates the sequence processing workflow. The next step 402, involves determining whether the genome sequence meets a predefined threshold. The threshold may consist of the pangenome and duplication indices plus some additional metrics such as sequence length and complexity (as measured by k-mer spectra or topological entropy). As one skilled artisan would appreciate that a k-mer spectrum, which is a representation of the k-mer content of sequencing data or genome, may estimate genome size and complexity, and show how many unique k-mers are present a certain number of times in the data. Whereas, topological entropy measures the complexity and randomness of a DNA sequence.

This decision point at step 402 is for filtering sequences that are suitable for further analysis. If the sequence does not meet the threshold, the process loops back to select another genome sequence from the reference database 300-N, ensuring only relevant sequences are processed further. If, however, the genome sequence meets the threshold, the method proceeds to step 403, where the sequence is processed with novel metrics, including a pangenome index and a duplication index. These metrics provide an analysis of genetic diversity by capturing both shared and unique genomic regions. The pangenome index allows for the integration of sequences from diverse individuals into a unified structure, facilitating rapid searches and analysis of genetic variations. The duplication index quantifies gene duplications, offering insights into evolutionary and functional consequences.

Following this processing, step 404 involves generating a set of scores by a machine learning algorithm for the genome sequence based on the novel metrics noted under step 403. The set of scores generated at this step 404 can constitute the following metrics, percentages, thresholds, ranges, etc. These generated set of scores are then transmitted back to the reference database 400-N for storage, contributing to the refinement of the database. One of the reference database 400-N may include an unfiltered source database which contains all genome sequences and one or more filtered databases that may contain a subset of the genome sequences in the source database (with inclusion determined by taxonomic origin of the sequence and the novel metrics).

The final step 405, involves continuously updating and refining the threshold based on classifications generated using the reference database 300-N, wherein human researchers may alter thresholds to change sensitivity and specificity as discussed above. Such continuous updating may be provided by a human expert via a device such that the threshold may be updated in real time. During development the updating of the thresholds may be a frequent process that might occur daily. Once a specific version of the tool is validated the thresholds and database content may generally remain fixed and unchanged. However, new versions of the tool are constantly being generated, but the only specific trigger for when the thresholds are altered is when some results that yield poor performance. This dynamic adjustment ensures that the method remains adaptive and improves over time. The flowchart of FIG. 4 effectively demonstrates the interaction between the various components, highlighting the ability to organize and represent genetic diversity through innovative metrics and machine learning techniques. The structural relationships and interactions between the steps ensure a cohesive process that enhances the accuracy and efficiency of genomic analysis.

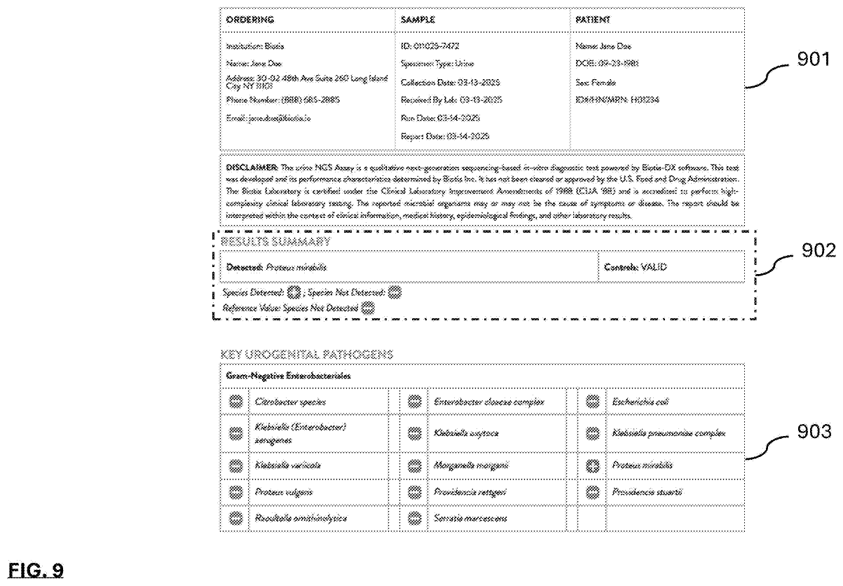

FIG. 5 illustrates a flowchart detailing a method for generating and processing classification databases using a machine learning algorithm. The steps set forth below in FIG. 5 may be performed in part by one or more processors executing computer readable instructions. The process begins with generating a coarse classification database 501 that includes a set of candidate microbial species. This initial step may involve rapid classification techniques to identify potential species present in the sample. The coarse classification database 501 is given at runtime, and it may be generated by the process described in steps 401-405 of FIG. 4.

A coarse classification database organizes data into broader, more general categories compared to fine-grained classifications. It groups similar items or concepts into higher-level classes, simplifying complex datasets and reducing dimensionality. This approach is useful for various applications, including protein structure analysis and data visualization. Examples of coarse classification databases include the Structural Classification of Proteins (SCOP) database, which organizes protein structures into hierarchical levels such as families, superfamilies, and folds. Another example is the CIFAR-100 dataset, which groups 100 fine-grained image classes into 20 super-classes or coarse categories. In the field of machine learning, coarse classification is often used as an intermediate step in hierarchical image classification models, where images are first classified into broader categories before being further refined into more specific classes.

Following this, a fine classification database 502 is generated, which comprises statistics and measures derived from alignments of sequencing reads to a microbial genome. In transitioning from step 501 to step 502 reads from the sample are mapped to the coarse classification database 501, wherein the result is a set of candidate organisms.

Step 502 can involve more precise alignment metrics and statistical analyses to refine the initial classification. A fine classification database in biotechnology organizes biological data into highly specific and detailed categories, allowing for precise differentiation between closely related entities such as genes, proteins, or organisms. These databases enable researchers to identify subtle differences and make granular distinctions, which is crucial for advanced genomic and proteomic studies. Examples of fine classification databases in biotechnology include the Structural Classification of Proteins (SCOP) database, which organizes protein structures into detailed hierarchical levels. The miRBase database focuses specifically on microRNA sequences, providing a fine-grained classification of these non-coding RNAs. Another example is the snoRNA-LBME-db, which stores small nucleolar RNA sequences and their targets, offering a highly specialized repository for this specific type of RNA.

Further, in step 502 the list of candidate organisms is used to generate a fine classification database 502. And, wherein this database is based on the output of the process described in steps 401-405 of FIG. 4, but is filtered for only those organisms identified as candidates. In proceeding from step 502 to step 503, reads are mapped to the fine classification database 502; and a set of measures and statistics are created for each candidate organism from these alignments.

The next step 503 involves processing a set of measures and statistics using the machine learning algorithm. The machine learning algorithm may be trained on data with known classifications, allowing it to generate a score (504) that reflects the likelihood or confidence of the presence of specific microbial species. The machine learning model may incorporate human-tunable hyperparameters, which can be iteratively refined by experts to enhance accuracy and reliability. During development or processing, the output from step 504 may be compared to known results for samples. This is used to calculate an overall sensitivity and specificity, and such numbers may be used by human researchers to set the thresholds discussed in FIG. 5.

Once the score is generated, it is used to update a results summary section of a report 505. This report may be structured to include both human-readable formats, such as PDFs, and machine-readable formats, like CSV or JSON, which can be accessed via an Application Programming Interface (API). The scores generated by a machine learning algorithm may be stored in a JSON file on either a local computer or an external server. Such results are uploaded to a lab information management system (LIMS), which triggers a service to generate a report. In other words, uploading the results to LIMS triggers generation of a report. This service downloads the JSON from the LIMS as well as patient information (name, age, sex, etc.) and inserts the data into a template. Thereafter, the service uploads the PDF report, which includes the results and patient information, back to the LIMS where it is flagged for review by a technician.

The updated report is then transmitted to a technician for approval 506, ensuring that the results are verified before being sent to a physician 507 for clinical decision-making. This process highlights the integration of machine learning with expert oversight to improve the accuracy and utility of microbial classification in clinical settings.

The flowchart in FIG. 5 describes a method involving the generation and refinement of classification databases and the use of machine learning for scoring. The described method may enhance the detection and classification of microbial species by leveraging both computational and expert-driven processes, ultimately leading to improved clinical outcomes. The use of a pangenome index and duplication index may further support the accuracy and comprehensiveness of the analysis by providing a representation of genetic diversity and duplication events.

Referring now to FIG. 6, the flowchart illustrates a detailed process for generating a classification database specifically for antimicrobial resistance genes and generating a database of reference variants. Overall, a human researcher may curate a set of gene variants that are associated with drug resistance (or other phenotype of interest). These gene variants may be obtained by literature review or by application of the tertiary index, wherein, the tertiary index is not used with the duplication index and pangenome index. The pangenome and duplication indices are used to construct a refence database for taxonomic identification. Whereas, the tertiary index is used to associate a variant with a phenotype. A researcher may use a database of reference genomes with known phenotype to validate or discover relevant variants.

In this process, a researcher may review and investigate literature and public databases to identify a set of genes and gene variants associated with resistance (or some other phenotype). Thereafter, the researcher may validate these variants by checking them against a database of genomes with known phenotypes; and if the researcher decides the variants are valid and useful they are included in the output database of resistance associated variants.

Referring back to FIG. 6, the process begins with step 601, where a classification database is generated. This database includes a set of statistics and measures derived from the alignment of sequencing reads to 3D protein structures of antimicrobial resistance genes. This step lays the foundation for subsequent analysis by providing a structured dataset that captures the genetic variations relevant to drug resistance. The database consists of a set of genomes with known phenotype for drug resistance. Wherein, these genomes consist of a set of genes and in turn the genes consist of a base sequence and a set of variants compared that sequence.

In step 602, the classification database is processed for generating a tertiary index. This may be done using a machine learning algorithm. This algorithm is designed to analyze the compiled statistics and measures, leveraging its training on similar datasets with known classifications to generate a score in step 603. The score reflects the likelihood or degree of antimicrobial resistance, providing a quantitative measure that can be used to inform clinical decisions.

Following the generation of the score, step 604 involves outputting a report of generating the tertiary index. This report is then transmitted to a technician for approval in step 605. The technician's role is to review the findings and ensure their accuracy and relevance before the report is forwarded to a physician. Alternatively, the report is transmitted to a human researcher who makes a decision about whether variants should or should not be included in reference set (based off of the output of the tertiary index and external factors such as literature review).

The flowchart in FIG. 6 highlights the integration of computational techniques, such as machine learning and 3D protein structure prediction, to enhance the accuracy of antimicrobial resistance classification. By converting nucleotide sequence data into actionable insights regarding microbial phenotypes, the process supports the selection of appropriate antimicrobials, ultimately aiming to improve patient health outcomes. The use of both short-read and long-read sequencing data, annotated with known species presence, further enriches the analysis, allowing for a robust and precise classification of resistance genes. This approach underscores the potential of combining genomic data with machine learning to address challenges in healthcare, particularly in the realm of infectious diseases and antimicrobial resistance.

Additionally, even with a set of variants obtained by the above noted process of FIG. 6, the resistance and phenotype may be difficult to determine. A researcher may use the variants output along with a database of genomes and phenotypes to develop a machine language classifier for phenotype. The researcher trains a machine learning classifier based on the set of reference variants from the above noted process and their database of genomes and phenotypes. In the event such classifier is too sensitive or specific, or insufficiently so the researcher may adjust criteria for inclusion of variants from the above noted process.

According to an example embodiment, a set of variants may be generated from the sample and reference (typically by aligning reads to reference genes); the variants are filtered to those matching process discussed above; the machine language classifier is used to predict phenotype from the sample; and the predicted phenotype is put into a report, approved by a technician, and then transmitted to a physician.

Referring now to FIG. 7, the flowchart illustrates a relearning algorithm process for a machine learning system, as implemented in FIGS. 5 and 6, which may be integral to the method for organizing and representing genetic diversity. The process begins with step 701, where training data is based on validated data sets. This initial phase may involve the application of numerous validated data sets to the machine learning algorithm, potentially validated by domain experts, to ensure the machine learning model is grounded in accurate and reliable data. This step is for establishing a foundation for the algorithm, allowing it to effectively process genome sequences with novel metrics such as the pangenome index and duplication index.

Following the initial training, step 702 involves reinforced learning by the machine learning algorithm. In this phase, the algorithm may be updated based on new data, including feedback from users or other input, to refine its performance. This iterative learning technique allows the algorithm to adjust in real-time, enhancing its ability to generate accurate scores for processed genome sequences. The reinforced learning phase may also involve the adjustment of weights and connections between nodes, enabling the algorithm to better identify patterns and relationships within the data.

The process concludes with step 703, which involves autonomous learning by the machine learning algorithm. This phase represents the algorithm's ability to continue learning independently, adapting to new data and refining its classifications without direct human intervention. The autonomous learning capability is essential for maintaining the algorithm's accuracy and relevance over time, particularly as it processes diverse genome sequences and updates the threshold based on classifications generated in the reference database.

The flowchart in FIG. 7 highlights the dynamic and iterative nature of the machine learning process, which is central to the ability to organize and represent genetic diversity. By employing a combination of training, reinforced learning, and autonomous learning, the machine learning algorithm can effectively support the generation of scores for genome sequences, contributing to the representation of genetic diversity through the pangenome index and duplication index. This process ultimately enhances the system's ability to perform variant detection, read alignment, and evolutionary studies.

The systems and methods of the inventive concepts disclosed herein implement a machine learning algorithm to convert nucleotide sequence data into classification of microbial species using machine learning models to implement a probabilistic determination, e.g., using a Bayesian, random forest, decision tree, linear regression, deep learning, neural network and/or dimensionality reduction techniques, as examples. The processing circuitry of the apparatus may have a graphics processing unit (GPU) and central processing unit (CPU) architecture, which may allow the apparatus to implement the machine learning algorithm. The machine learning algorithm and the GPU architecture also enable the apparatus to make the probabilistic determination by considering large amounts of diverse data together and identifying patterns in the data. The data may include present and historical values of (or values derived from). In some examples, a result of the application of the machine learning algorithm accurately identifies not only the causative pathogen but virulence factors and antimicrobial resistance to guide precision infectious disease treatment.

Referring now to FIG. 8, the system architecture for organizing and representing genetic diversity is depicted. The system 802 comprises a central processing unit (CPU) 803, memory 805, a storage unit 806, and a machine learning algorithm 807. These components are interconnected via a communication bus 808, facilitating data transfer and processing. The CPU 803, which may include one or more processors and/or one or more graphics processing units (GPUs), executes machine-readable instructions stored in the memory 805 or storage unit 806 (e.g., hard disk), enabling the system to perform genomic analyses. Examples of operations performed by the CPU 803 can include fetch, decode, execute, and writeback. The CPU 803 can be part of a circuit, such as an integrated circuit. One or more other components of the system 802 can be included in the circuit. In some cases, the circuit is an application specific integrated circuit (ASIC).

The memory 805 can include various types of memory such as RAM, ROM, or flash memory, providing both volatile and non-volatile storage options. The storage unit 806 serves as a repository for data, including user preferences, programs, and genomic data, which may be stored locally or accessed from remote servers.

The system 802 is connected to multiple reference databases 300-1, 300-2, through 300-N via a network 304, which may be the Internet or an intranet. The network 304 in some cases is a telecommunication and/or data network. The network 304 can include one or more computer servers, which can enable distributed computing, such as cloud computing. This network connection, facilitated by the communication interface 804, allows for distributed data storage and access, enabling the system to retrieve and process genome sequences from diverse sources. The network 304 supports cloud computing capabilities, allowing for scalable and efficient data processing and analysis. For example, one or more computer servers may enable cloud computing over the network 304 (“the cloud”) to perform various aspects of analysis, calculation, and generation of the present disclosure, such as, for example, (i) train and test a trained algorithm, (ii) generate a reference database of microbial genomes optimized for detection of species in clinical samples; (iii) convert nucleotide sequence data into classification of microbial species using machine learning models, and optimized for clinical assay; (iv) convert nucleotide sequence data into classification of microbial phenotype for drug resistance by implementing 3D protein structure prediction to categorize mutation that aid in guiding selection of antimicrobials and improving patient outcomes; (v) use the trained algorithm to process data indicative of a presence, absence, or relative assessment of identifying and characterizing pathogens, including antimicrobial resistance and virulence factors in clinical specimens; and (vi) electronically output a report that identifies or provides an indication of the urologic condition of the subject. Such cloud computing may be provided by cloud computing platforms such as, for example, Amazon Web Services (AWS), Microsoft Azure, Google Cloud Platform, and IBM cloud. The network 304, in some cases with the aid of the computer system 802, can implement a peer-to-peer network, which may enable devices coupled to the computer system 802 to behave as a client or a server.

The machine learning algorithm 807 processes genome sequences using novel metrics such as the pangenome index and duplication index, generating scores that are transmitted back to the reference databases for storage and further analysis. The memory 805 with computer readable instructions stored thereon may be configured to store one or more algorithms. For example, an algorithm may comprise a machine learning algorithm 807. The machine learning algorithm 807 may be configured to identify and characterize pathogens, including antimicrobial resistance and virulence factors in clinical specimens.

In an example, the machine learning algorithm 807 is applied via one or more processors that are configured to execute computer readable instructions. The machine learning algorithm 807 may be part of a computer system 802, or alternatively, an external server (not shown) that is in communication with the computer system 802. The machine learning algorithm 807 may be automatically updated, e.g., continue to learn, based on new data stored in memory 805 and/or reference database 300-N. In general, the data may include values that vary over time, and the machine learning algorithm 807 may consider various factors including receiving feedback from a user that may also pertain to false positives that allow the machine learning algorithm 807 to adjust in real-time. Adjustments to the machine learning algorithm 807 may include adjusting weights and/or connections between nodes, as examples.

Based on learning from sets of data, the machine learning algorithm 807 may discover patterns in, and relationships, between several independent and interdependent variables derivable from the data. Some of these independent and interdependent variables derivable from the data may include, GeneCoverage; ReadIdentity; CountRepeatKmers; GCBias; and AssemblyStats. ReadIdentity may correspond to the percentage of identity reads from a sample to a reference; GeneCoverage may correspond to what percentage of the reference genes are covered by a sample's reads; CountRepeatKmers may correspond to the fraction of kmers in the sample's reads that map to more than one reference genome; GCBias may correspond to how much of the sample's reads that map to the genome are composed of the nucleotides G and C vs A and T; and AssemblyStats may correspond to various stats about how many and how long assemblies from a given sample could be generated mapping to a specific organism.

Ongoing consideration of these variables, or other learned variables from continued updating of the algorithm, may allow the machine learning algorithm to probabilistically determine, e.g., classify, diagnose, and/or predict, a state of the patient. The machine learning algorithm may be configured to employ any one or more of Bayesian, random forest, decision tree, linear regression, deep learning, neural network and/or dimensionality reduction techniques, as examples. In some examples, a result of the application of the machine learning algorithm accurately identifies not only the causative pathogen but virulence factors and antimicrobial resistance to guide precision infectious disease treatment.

Still referring to FIG. 8, peripheral devices 809, such as electronic display adapters, are connected to the system via the communication bus 808, providing user interfaces for data visualization and interaction. The electronic display device 801, connected to the system, may present graphical user interfaces (GUIs) or web-based interfaces, allowing users to interact with the system, view analysis results, and generate reports. This setup supports various applications, including the classification of microbial species, detection of antimicrobial resistance, and evolutionary studies.

The computer system 802 can communicate with one or more remote computer systems through the network 304. For instance, the computer system 802 can communicate with a remote computer system of a user such as electronic display device 801. Examples of remote computer systems include personal computers (e.g., portable PC), slate or tablet PC's (e.g., Apple® iPad, Samsung® Galaxy Tab), telephones, Smart phones (e.g., Apple® iphone, Android-enabled device, Blackberry®), or personal digital assistants. The user can access the computer system 802 via the network 304.

Methods as described herein can be implemented by way of machine (e.g., computer processor) executable code stored on an electronic storage location of the computer system 802, such as, for example, on the memory 805 or electronic storage unit 806. The machine executable or machine-readable code can be provided in the form of software. During use, the code can be executed by the processor 803. In some cases, the code can be retrieved from the storage unit 806 and stored on the memory 805 for ready access by the processor 803. In some situations, the electronic storage unit 806 can be precluded, and machine-executable instructions are stored on memory 805.

The code can be pre-compiled and configured for use with a machine having a processer adapted to execute the code, or can be compiled during runtime. The code can be supplied in a programming language that can be selected to enable the code to execute in a pre compiled or as-compiled fashion.

Aspects of the systems and methods provided herein, such as the computer system 802, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 802 is programmed or otherwise configured to, for example, (i) train and test a trained algorithm, (ii) generate a reference database of microbial genomes optimized for detection of species in clinical samples; (iii) convert nucleotide sequence data into classification of microbial species using machine learning models, and optimized for clinical assay; (iv) convert nucleotide sequence data into classification of microbial phenotype for drug resistance by implementing 3D protein structure prediction to categorize mutation that aid in guiding selection of antimicrobials and improving patient outcomes; (v) use the trained algorithm to process data indicative of a presence, absence, or relative assessment of identifying and characterizing pathogens, including antimicrobial resistance and virulence factors in clinical specimens; and (vi) electronically output a report that identifies or provides an indication of the urologic condition of the subject.

The system architecture is designed to support the continuous updating and refining of thresholds based on classifications generated in the reference databases, enhancing the accuracy and reliability of genomic analyses. The integration of the pangenome index and duplication index within the machine learning framework allows for a representation of genetic diversity, facilitating rapid searches and analysis of genetic variations. This architecture is optimized for clinical and research applications, providing insights into genetic diversity and aiding in the development of targeted treatments and interventions.

Referring now to FIG. 9, the report format is designed to display patient information, a results summary, and key urogenital pathogens, which may be used in clinical settings for reporting diagnostic test results. The first section labeled ORDERING, SAMPLE, PATIENT 901 includes details about the institution, patient, and sample. This section may contain the institution's name, patient identification, specimen type, and collection details, providing background information for the diagnostic process. The inclusion of such information ensures that the report is tailored to the specific patient and sample, facilitating interpretation and follow-up actions.

The second section is the RESULTS SUMMARY 902 section, which provides information on detected species and control validation. This part of the report is generated by a machine learning algorithm, which may optimize genomics and machine learning-based assays to identify and characterize pathogens, including antimicrobial resistance and virulence factors in clinical specimens. The results summary section 902 may indicate whether a specific pathogen, such as Proteus mirabilis, has been detected, and it validates the controls used in the assay. This automated generation of results ensures consistency and accuracy, reducing the potential for human error in the interpretation of complex genomic data.

Next, the third section is the KEY UROGENITAL PATHOGENS (903) section lists various pathogens, highlighting detected species. This section may include a range of gram-negative enterobacteria, with specific pathogens marked to indicate detection. The structured presentation of pathogens allows for quick reference and aids healthcare professionals in understanding the potential clinical implications of the test results. The report format, as depicted, may be electronically transmitted to a technician for approval before being sent to a physician for review, ensuring a streamlined workflow in clinical diagnostics. This integration of machine learning and genomic analysis into the reporting process exemplifies the capabilities of the system in managing and interpreting genetic diversity data.

As discussed above, the report shown in FIG. 9 may be structured to include both human-readable formats, such as PDFs, and machine-readable formats, like CSV or JSON, which can be accessed via an Application Programming Interface (API).

The scores generated by a machine learning algorithm may be stored in a JSON file on either a local computer or an external server. Upon processing of computer readable instructions and selection of input on a computer screen, such results are uploaded to a lab information management system (LIMS), which triggers a service to generate a report. In other words, uploading the results to LIMS triggers generation of a report. LIMS may be another software component that is configured to run on a computer system or an external server. Thereafter, JSON is downloaded from the LIMS and internally modified in the computer system or an external server to insert data. In other words, the addition of data is not performed by a human or a researcher. But, instead, the report is modified by a computer system or a server to add additional data thereto by inserting data into it that was not previously present. The modification of the report by the computer system or server is performed and thereafter transmitted back to the LIMS where it is flagged for review by a technician. The flagging for review may correspond the technician receiving an email notification or an alert notification on his/her phone, tablet, iPad, personal computer, desktop etc.

To summarize, the modifying of the report and adding of data thereto is performed by a computer system or a server and not by a human. And, thereafter, such report with the added data is transmitted over a network to a technician for review and further analysis.

### IV. Uses of an Assay of the Disclosure

### Next-Generation Sequencing-Based Assay for Antimicrobial Resistance Gene Marker Identification in Urine

In some aspects the disclosure can be applied to the detection of antimicrobial resistance (AMR) genes in clinical specimens using next-generation sequencing (NGS) techniques. The constant increase in antimicrobial resistance (AMR) represents a worsening global threat, contributing to approximately 1.27 million deaths annually. Traditional culture and phenotypic resistance testing are considered the gold standard for diagnosing urinary tract infections (UTIs). However, these methods have limitations, including false-negative results and long turnaround times, which can lead to inconclusive results and empirical treatment. Polymerase chain reaction (PCR) is a fast and sensitive molecular diagnostic method but is limited in covering the robust number of antibiotic resistance genes (ARGs). In some aspects the disclosure provides an assay for the detection of antimicrobial resistance (AMR) genes in clinical specimens using next-generation sequencing (NGS) techniques. The invention provides a method and system for identifying uropathogens and profiling drug resistance in urinary tract infections (UTIs).

### EXAMPLES

The present technology is not to be limited in terms of the particular implementations described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular implementations only, and is not intended to be limiting.

### Example 1: Validation of Urine NGS Assay

This example details the validation of the disclosed urine NGS assay described herein.

BIOTIA-ID Urine NGS Assay was evaluated on a combination of urine clinical specimens and contrived spike-in samples using whole organism microbial reference strains and clinical isolates. In this study, 954 samples were processed at the laboratory (143 clinical specimens, 811 contrived) and tested with BIOTIA-ID Urine NGS Assay in seven separate studies. The BIOTIA-ID Urine NGS Assay validation yielded an overall assay performance of 99.89% sensitivity and 99.94% specificity when evaluating the entire validation dataset (n=9,441 analytes).

Whole organisms of key urogenital pathogens detected by our assay, genetically related organisms, and other organisms that can be present in urine specimens were included in the specificity studies. A total of 64 microbial species (bacteria, fungi, viruses, and parasites) and 119 total strains were evaluated in the laboratory. Whole organisms were spiked into negative urine matrix and processed with the assay. The specificity study entailed testing contrived specimens which resulted in an overall sensitivity and specificity of 100% and 99.95%.

We also conducted an extensive in silico analysis to further evaluate the performance and specificity of the assay. A total of 8,266 of simulated samples were processed and tested for key organisms. An overall sensitivity of 99.96% and specificity of 99.95% was obtained for simulated samples.

The sensitivity studies sought to determine the limit of detection (LoD) of our assay in ten urogenital pathogens (A. baumanii, B. fragilis, E. coli, E. faecalis, G. vaginalis, K. pneumoniae, Prevotella spp., P. mirabilis, P. aeruginosa, and S. aureus). Whole organisms were spiked into negative urine matrix at different CFU/mL concentrations and 6 total replicates were tested per concentration. A total of 495 contrived specimens were processed for this component of the validation. The LoD was determined as the lowest concentration in which 100% of the replicates (6/6) were detected. The overall LoD was <25,000 colony-forming units per milliliter (CFU/mL). Specifically, the LoDs determined for each species were 7,500 CFU/mL for E. coli, G. vaginalis, K. pneumoniae and P. mirabilis; 10,000 CFU/mL for E. faecalis; 12,500 CFU/mL for Prevotella spp.; 15,000 CFU/mL for A. baumanii and S. aureus; 25,000 CFU/mL for P. aeruginosa and B. fragilis.

Finally, an inter/intra-assay reproducibility study was used to assess variability in the assay. Five different pools containing one clinical strain of E. coli, E. faecalis, K. pneumoniae, P. mirabilis and S. aureus were tested. Each pool had different strains for each species (altogether 25 different strains) and were spiked into negative urine matrix at a concentration of 25,000 CFU/mL per analyte. Subsequently, each pool was tested daily for three consecutive days in three technical replicates for a total of 15 replicates per pool, 45 replicates per analyte in addition to 6 negative samples. A follow up study, sought to evaluate reproducibility of detection at different concentrations: 100,000 CFU/mL (high); 25,000 CFU/mL and 20,000 CFU/mL (medium); and 15,000 CFU/mL (low). Two bacterial pools per concentration were tested in triplicates, three different days for a total of 9 replicates per concentration per analyte. A total of 36 replicates per analyte were evaluated, yielding replicable results for all concentrations and species tested in this second study. Both studies reproducibly and correctly detected each sample tested obtaining a sensitivity and specificity of 100% (n=81/81 observations per analyte).

The accuracy verification study was comprised of 190 total specimens (47 contrived, 143 urine clinical specimens). Urine clinical samples were collected based on the pathogens diagnosed by culture and were comprised of five of the most common urogenital pathogens (E. coli, E. faecalis, K. pneumoniae, P. mirabilis and S. aureus). We collected at least 30 specimens per analyte and used contrived specimens when clinical specimens were not available. This study was a randomized, double blinded study. The clinical validation yielded an overall performance of 97.35% sensitivity and 99.65% specificity.

### Validation Sample Summary

Table 1 shows a summary of specimens processed in each study of the validation.

TABLE 1
Summary of Specimens Process in Each Study of the Validation
Table 1

| Sample | | | | Accuracy | |
|---|---|---|---|---|---|
| Type | Specificity | Sensitivity | Reproducibility | Verification | Total |
| Clinical | — | — | — | 143 | 143 |
| Contrived | 119 | 495 | 150 | 47 | 811 |
| In silico | 8266 | — | — | — | 8266 |
| NEC | 10 | 28 | 17 | 20 | 75 |
| NTC | 5 | 22 | 13 | 10 | 50 |
| PC | 5 | 22 | 13 | 10 | 50 |
| Total | 8400 | 545 | 180 | 77 | 9395 |

### Laboratory Specificity Experiments

Reference organisms and clinical isolates were tested and validated on the specificity component of the BIOTIA-ID Urine NGS Assay clinical validation. Whole organisms of key urogenital pathogens detected by the assay, genetically related organisms, and other organisms that can be present in urine specimens were tested by spiking microbial cells into negative urine matrix. A total of 64 microbial species (bacteria, fungi, viruses and parasites) and 119 strains were evaluated. Microbial isolates were spiked into negative urine matrix at a concentration of 50,000 CFU/mL and processed with the BIOTIA-ID Urine NGS Assay resulting in an overall sensitivity and specificity of 100% and 99.95%. A detailed summary of sensitivity and specificity for all microbial species tested can be found in Table 3 below and in supplement.

At clinically relevant analyte concentrations, the following possible cross-reactions were observed with closely related strains, or other analytes of the BIOTIA-ID Urine NGS Assay:
Possible cross-reaction of Shigella species with the analyte E. coli. Shigella species are not reportable organisms on the BIOTIA-ID Urine NGS Assay and are not a common urogenital pathogen.
Possible cross-reaction of K. variicola with the analyte K. pneumoniae.
We also found cross-reactivity of HPV with the analyte E. coli; however, we believe this is due to how this control is manufactured. This control is a recombinant control that is produced in E. coli. We confirmed presence of E coli on this control by testing two aliquots from the same lot with the E. coli uidA comparator testing qPCR assay.

### In Silico Cross-Reactivity

To analyze cross-reactivity in silico a total of 8,266 RefSeq genomes comprised from 5,340 unique bacteria, eukaryotic organisms, and viruses were used to simulate fastq files with 100,000 reads each. A random mutation rate of 0.5% was applied during simulation to introduce a reasonable degree of variability. Simulated read files were then run through BIOTIA-DX in an identical manner to sequenced laboratory samples and tested for key organisms. In this way, we could assess the potential for organisms outside our laboratory sample set to generate false positive results for key organisms. Overall, the sensitivity and specificity of in silico samples is 99.96% and 99.95%, respectively. This study showed few cross-reactive samples (n=147, 1.7% of all synthetic samples), which are summarized in Table 3 below and in supplementary material. The majority of the cross-reactive species observed were either within the same genus, do not have clinical relevance or are unlikely to be found in a urine specimen. However, some of the observed cross-reactivities are with species within the same species complex. For such cases, we will report them as the complex instead of the individual species (e.g., Acinetobacter calcoaceticus-baumannii complex (ACB complex), Enterobacter cloacae complex, and Klebsiella pneumoniae complex).

The original in silico validation strategy included three different genomes from NCBI GenBank for Providencia rettgeri from which simulated sequencing files were generated using a mutation rate of 0.005 and an error rate of 0.01. These simulated files were then run through BIOTIA-DX. Only 2 of the 3 genomes used for the P. rettgeri simulations resulted in positive identifications by BIOTIA-DX, resulting in a sensitivity of ⅔ or 67.7%. Upon further analysis, we discovered that the failed genome is not in fact Providencia rettgeri at all, but instead is a misidentified strain of Proteus vulgaris incorrectly annotated in GenBank, which explains why it was missed by BIOTIA-DX. As part of this resubmission, we have included a new Providencia rettgeri simulation using one of the two successful genomes from the original validation which we simulated at double the original mutation rate (0.01). In this case, BIOTIA-DX correctly identified the simulated sample as Providencia rettgeri with a prediction probability of 0.988, which would bring the sensitivity to 3/3 or 100%.

### Specificity Performance Characteristics

Table 2 shows a summary of performance characteristics of a specificity study

TABLE 2
Table 2: Summary of Performance Characteristics
of Specificity Study (contrived and in silico)

| Specificity | TP | TN | FN | FP | Total | Sensitivity | Specificity |
|---|---|---|---|---|---|---|---|
| Contrived | 119 | 4044 | 0 | 2 | 4165 | 100.00% | 99.95% |
| In silico | 8119 | 272775 | 3 | 147 | 281044 | 99.96% | 99.95% |

Table 3 shows a summary of in silico cross-reactive species obtained with the BIOTIA-DX software. Asterisks (*) indicate species that showed cross-reactivity but belong to the same species complex.

TABLE 3
Summary of in silico cross-reactive species
obtained with BIOTIA-DX software.
Table 3

| | # of | |
|---|---|---|
| Analyte | samples | Cross reactive species |
| Acinetobacter baumanii | 19 | Acinetobacter calcoaceticus* |
| | | Acinetobacter genomosp |
| | | Acinetobacter lactucae |
| | | Acinetobacter nosocomialis* |
| | | Acinetobacter pitti* |
| | | Acinetobacter seifertii |
| | | Acinetobacter oleivorans |
| Acinetobacter lwoffii | 3 | Acinetobacter pseudolwoffii |
| Enterobacter cloacae | 10 | Enterobacter asburiae* |
| | | Enterobacter chengduensis |
| | | Enterobacter cloacae complex* |
| | | Enterobacter genomosp |
| | | Enterobacter kobei* |
| | | Enterobacter quasiroggenkampii |
| | | Enterobacter sichuanensis |
| Enterococcus faecium | 3 | Enterococcus lactis |
| Escherichia coli | 48 | Escherichia albertii |
| | | Escherichia marmotae |
| | | Escherichia fergusonii |
| | | Shigella boydii |
| | | Shigella flexneri |
| | | Shigella sonnei |
| | | Shigella dysenteriae |
| Klebsiella oxytoca | 3 | Klebsiella grimontii |
| Klebsiella pneumoniae | 13 | Klebsiella variicola* |
| | | Klebsiella quasipneumoniae* |
| | | Klebsiella quasivariicola* |
| | | Klebsiella africana |
| Klebsiella variicola | 3 | Klebsiella quasipneumoniae |
| | | Klebsiella quasivariicola |
| Morganella morganii | 1 | Morganella psychotolerans |
| Proteus mirabilis | 8 | Proteus columbae |
| | | Proteus penneri |
| | | Proteus terrae |
| | | Proteus vulgaris |
| Proteus vulgaris | 7 | Proteus columbae |
| | | Proteus faecis |
| | | Proteus terrae |
| Providencia rettgeri | 3 | Providencia huaxiensis |
| Providencia stuartii | 3 | Providencia thailandensis |
| Pseudomonas aeruginosa | 3 | Pseudomonas paraeruginosa |
| Raoultella ornithinolytica | 3 | Raoultella planticola |
| Serratia marcescens | 18 | Serratia bockelmannii |
| | | Serratia nematodiphila |
| | | Serratia nevei |
| | | Serratia surfactantfaciens |
| | | Serratia ureilytica |
| Staphylococcus aureus | 11 | Staphylococcus argenteus |
| | | Staphylococcus roterodami |
| | | Staphylococcus schweitzeri |
| | | Staphylococcus singaporensis |
| Stenotrophomonas | 11 | Stenotrophomonas geniculata |
| maltophilia | | Stenotrophomonas indicatrix |
| | | Stenotrophomonas lactitubi |
| | | Stenotrophomonas pavanii |
| Streptococcus mitis | 1 | Streptococcus pseudopneumoniae |

### Sensitivity

The sensitivity studies established the limit of detection (LoD) of our assay in ten urogenital pathogens (A. baumanii, B. fragilis, E. coli, E. faecalis, G. vaginalis, K. pneumoniae, Prevotella spp., P. mirabilis, P. aeruginosa, and S. aureus). The LoD was determined by performing a 10-fold dilution series of whole organisms of interest (6 data points, 1E+01 to 1E+06 CFU/mL) and spiking these in triplicates into negative clinical urine matrix. Each LoD experiment was conducted in duplicates for 6 total replicates per concentration, per analyte tested. After obtaining the lowest detectable concentration in the 10-fold dilution series experiments, a dilution series from 1E+01 to 5E+04 was used to fine-tune the LoD. A total of 495 contrived samples were tested in this study. The LoD was established when target organism is detected in 95% of the samples which is equal to 6/6 replicates.

First, the LoD was assessed on five of the most prevalent urogenital pathogens, (E. coli, E. faecalis, K. pneumoniae, P. mirabilis and S. aureus) using clinical isolates spiked into negative urine matrix. The LoD was reproducibly verified and determined based on a 100% positivity rate using the assay. The overall LoD in this set of experiments was <10,000 CFU/mL. Specifically, the LoDs were determined as 1,000 CFU/mL for S. aureus; 7,500 CFU/mL for E. coli, K. pneumoniae and P. mirabilis; and 10,000 CFU/mL for E. faecalis (Table 6A below and in supplementary material).

Table 4 shows a summary of results from the analytical sensitivity study per analyte.

TABLE 4
Table 4: Summary of Results from the Analytical Sensitivity Study Per Analyte

| | Detection | | | | | | | | | | | |
|---|---|---|---|---|---|---|---|---|---|---|---|---|
| | Limit | CFU/mL | | | | | | | | | | |
| Analyte | (CFU/mL) | 10 | 1 × 10² | 1 × 10³ | 5 × 10³ | 7.5 × 10³ | 1 × 10⁴ | 1.25 × 10⁴ | 2.5 × 10⁴ | 5 × 10⁴ | 1 × 10⁵ | 1 × 10⁶ |
| Staphylococcus | 1,000** | 0/6 | 0/6 | 6/6 | — | — | 6/6 | — | 6/6 | — | 6/6 | 6/6 |
| aureus | | | | | | | | | | | | |
| Escherichia | 7,500 | 0/3 | 0/3 | 3/9 | 5/6 | 6/6 | 6/6 | 6/6 | 6/6 | 6/6 | 6/6 | 6/6 |
| coli | | | | | | | | | | | | |
| Klebsiella | 7,500 | 0/3 | 0/3 | 0/6 | 4/6 | 6/6 | 6/6 | 6/6 | 6/6 | 6/6 | 6/6 | 6/6 |
| pneumoniae | | | | | | | | | | | | |
| Proteus | 7,500 | 0/6 | 0/6 | 3/12 | 3/6 | 6/6 | 6/6 | 6/6 | 6/6 | 6/6 | 6/6 | 6/6 |
| mirabilis | | | | | | | | | | | | |
| Enterococcus | 10,000 | 0/3 | 0/3 | 3/6 | 0/6 | 4/6 | 6/6 | 6/6 | 6/6 | 6/6 | 6/6 | 6/6 |
| faecalis | | | | | | | | | | | | |

Further, in this revision, 5 new analytes were added to include organisms pertaining to the following groups: Gram Negative non Enterobacteriales (Acinetobacter baumanii, Pseudomonas aeruginosa); Anaerobic Bacteria (Prevotella species, Bacteroides fragilis) and Other Bacteria (Gardnerella vaginalis). In these experiments, analytes were pooled (3 species per sample) to account for variability in the presence of multiple organisms. Each analyte was tested as 6 replicates for every CFU/mL concentration evaluated in these experiments.

Table 5 shows a summary of sensitivity study of new analytes.

TABLE 5
Table 5: Summary of Sensitivity Study of New Analytes

| | Detection | | | | | | | | | | | |
|---|---|---|---|---|---|---|---|---|---|---|---|---|
| | Limit | CFU/mL | | | | | | | | | | |
| Analyte | (CFU/mL) | 10 | 1 × 10² | 1 × 10³ | 5 × 10³ | 7.5 × 10³ | 1 × 10⁴ | 1.25 × 10⁴ | 2.5 × 10⁴ | 5 × 10⁴ | 1 × 10⁵ | 1 × 10⁶ |
| Acinetobacter | 15,000* | — | 0/3 | 0/3 | 0/6 | 0/6 | 4/6 | 5/6 | 6/6 | 4/6 | 6/6 | 6/6 |
| baumanii | | | | | | | | | | | | |
| Pseudomonas | 25,500 | — | 0/3 | 0/3 | 0/6 | 0/6 | 4/6 | 5/6 | 6/6 | 6/6 | 6/6 | 6/6 |
| aeruginosa | | | | | | | | | | | | |
| Prevotella | 12,500 | — | 0/3 | 0/3 | 5/6 | 6/6 | 5/6 | 6/6 | 6/6 | 6/6 | 6/6 | 6/6 |
| species | | | | | | | | | | | | |
| Bacteroides | 25,000 | — | 0/3 | 0/3 | 0/6 | 1/6 | 1/6 | 4/6 | 6/6 | 6/6 | 6/6 | 6/6 |
| fragilis | | | | | | | | | | | | |
| Gardnerella | 7,500 | — | 0/3 | 0/3 | 4/6 | 6/6 | 6/6 | 6/6 | 6/6 | 6/6 | 6/6 | 6/6 |
| vaginalis | | | | | | | | | | | | |
| Staphylococcus | 15,000** | 0/6 | 0/6 | 6/6 | 2/3 | 3/3 | 6/6 | 2/6 | 6/6 | 6/6 | 6/6 | 6/6s |
| aureus | | | | | | | | | | | | |

To demonstrate whether presence of multiple analytes has no adverse effect on the LoD, two bacterial pools containing three organisms per pool were tested in triplicates, three different days for a total of 9 replicates per analyte. Spike in concentrations of 25,000 CFU/mL, 20,000 CFU/mL and 15,000 CFU/mL were evaluated in Escherichia coli, Klebsiella pneumoniae, Enterococcus faecalis, Proteus mirabilis, Acinetobacter baumanii and Staphylococcus aureus. All analytes were reproducibly detected with 100% sensitivity and specificity in every concentration tested. Results for these experiments are summarized in Table 6C below.

Table 6 shows sensitivity experiments with multiple analytes per sample.

TABLE 6
Sensitivity Experiments with Multiple Analytes Per Sample
Table 6

CFU/ml

| Analyte | 15,000 | 20,000 | 25,000 |
|---|---|---|---|
| Escherichia coli | 9/9 | 9/9 | 9/9 |
| Klebsiella pneumoniae | 9/9 | 9/9 | 9/9 |
| Enterococcus faecalis | 9/9 | 9/9 | 9/9 |
| Proteus mirabilis | 9/9 | 9/9 | 9/9 |
| Acinetobacter baumanii | 9/9 | 9/9 | 9/9 |
| Staphylococcus aureus | 9/9 | 9/9 | 9/9 |

The overall LoD was <25,000 CFU/mL, specifically, the LoDs established for each species was 7,500 CFU/mL for E. coli, G. vaginalis, K. pneumoniae and P. mirabilis; 10,000 CFU/mL for E. faecalis; 12,500 CFU/mL for Prevotella spp.; 15,000 CFU/mL for A. baumanii and S. aureus; 25,000 CFU/mL for P. aeruginosa and B. fragilis.

A technical oversight at the lab, led to unintentional omission of 3 replicates each for 5,000 and 7,500 CFU/mL. Additionally, lower dilution samples from S. aureus at 12,500 and 7,500 resulted in some replicates generating borderline prediction probabilities (0.43-0.45) that yielded false negative results with BIOTIA-DX (See Supplementary Table 19). A similar issue is observed for two samples for A. baumanii at 50,000 CFU/mL. However, results from experiments highlighted in Table 6C show that both S. aureus and A. baumanii were reproducibly detected at 15,000; 20,000 and 25,000 CFU/mL when tested with multiple analytes in the sample. Additionally, six more replicates of A. baumanii at 25,000 CFU/mL were tested in sensitivity experiments highlighted in Table 6B. These results could be possibly explained by technical error at the time of processing the samples possibly during library preparation and/or spike in of analytes. Therefore, we have established the LoD for these analytes at 15,000 CFU/mL.

### Inter/Intra-Assay Reproducibility

Inter/intra assay reproducibility were performed in two different studies. In the first study, five different pools containing one clinical strain each of E. coli, E. faecalis, K. pneumoniae, P. mirabilis and S. aureus were tested. Each pool had different strains for each species (altogether 25 different strains) and were spiked into negative urine matrix at a concentration of 25,000 CFU/mL per analyte. Subsequently, each pool was tested daily for three consecutive days in three technical replicates for a total of 15 replicates per pool), 45 replicates per analyte and 6 negative samples. All analytes were reproducibly and correctly detected in each sample tested, obtaining a sensitivity and specificity of 100% (Table 7 below and in supplement). Table 7 shows the number of samples in which the organism was detected over the number of samples tested in each pool (1-5) and day (1-3).

TABLE 7
Summary of Inter/Intra Assay Reproducibility Results per Analyte
Table 7

| Organism | Pool | Day One | Day Two | Day Three |
|---|---|---|---|---|
| Escherichia coli | 1 | 3/3 | 3/3 | 3/3 |
| | 2 | 3/3 | 3/3 | 3/3 |
| | 3 | 3/3 | 3/3 | 3/3 |
| | 4 | 3/3 | 3/3 | 3/3 |
| | 5 | 3/3 | 3/3 | 3/3 |
| Klebsiella pneumoniae | 1 | 3/3 | 3/3 | 3/3 |
| | 2 | 3/3 | 3/3 | 3/3 |
| | 3 | 3/3 | 3/3 | 3/3 |
| | 4 | 3/3 | 3/3 | 3/3 |
| | 5 | 3/3 | 3/3 | 3/3 |
| Proteus mirabilis | 1 | 3/3 | 3/3 | 3/3 |
| | 2 | 3/3 | 3/3 | 3/3 |
| | 3 | 3/3 | 3/3 | 3/3 |
| | 4 | 3/3 | 3/3 | 3/3 |
| | 5 | 3/3 | 3/3 | 3/3 |
| Enterococcus faecalis | 1 | 3/3 | 3/3 | 3/3 |
| | 2 | 3/3 | 3/3 | 3/3 |
| | 3 | 3/3 | 3/3 | 3/3 |
| | 4 | 3/3 | 3/3 | 3/3 |
| | 5 | 3/3 | 3/3 | 3/3 |
| Staphylococcus aureus | 1 | 3/3 | 3/3 | 3/3 |
| | 2 | 3/3 | 3/3 | 3/3 |
| | 3 | 3/3 | 3/3 | 3/3 |
| | 4 | 3/3 | 3/3 | 3/3 |
| | 5 | 3/3 | 3/3 | 3/3 |
| Negative Sample | — | 2/2 | 2/2 | 2/2 |

A follow up study, sought to evaluate reproducibility of detection at different concentrations: 100,000 CFU/mL (high); 25,000 CFU/mL and 20,000 CFU/mL (medium); and 15,000 CFU/mL (low) in Escherichia coli, Klebsiella pneumoniae, Proteus mirabilis, Enterococcus faecalis and Staphylococcus aureus. Two bacterial pools per concentration were tested in triplicates, three different days for a total of 9 replicates per concentration per analyte in addition to 9 negative samples. In these set of experiments, 36/36 replicates were detected per analyte with 180 total analytes evaluated which yielded consistent and replicable results for all concentrations and species tested. Results for these set of experiments are summarized in Table 7B below and in supplement.

Both inter and intra reproducibility studies correctly detected each sample evaluated with this assay (n=150) obtaining a sensitivity and specificity of 100%, regardless of the analyte concentration. These studies yielded 81/81 correct observations per analyte for a total of 420 analytes correctly detected with BIOTIA-ID Urine NGS Assay.

Table 8 shows inter and intra reproducibility at high, medium and low concentration.

TABLE 8
Table 8: Inter and Intra Reproducibility at High, Medium and Low Concentration

| Analyte | Strain | Load | Day 1 | Day 2 | Day 3 | Total | Analytes | Samples |
|---|---|---|---|---|---|---|---|---|
| Enterococcus | URV- | 25,000 | 15/15 | 15/15 | 15/15 | 45/45 | 225 | 51 |
| faecalis* | 220 | CFU/ml | | | | | | |
| | URV- | | | | | | | |
| | 032-2 | | | | | | | |
| | URV- | | | | | | | |
| | 007 | | | | | | | |
| | URV- | | | | | | | |
| | 026 | | | | | | | |
| | URV- | | | | | | | |
| | 261 | | | | | | | |
| Escherichia | URV- | 25,000 | 15/15 | 15/15 | 15/15 | 45/45 | | |
| coli* | 005-1 | CFU/ml | | | | | | |
| | URV- | | | | | | | |
| | 012 | | | | | | | |
| | URV- | | | | | | | |
| | 029 | | | | | | | |
| | URV- | | | | | | | |
| | 006 | | | | | | | |
| | URV- | | | | | | | |
| | 021 | | | | | | | |
| Klebsiella | URV- | 25,000 | 15/15 | 15/15 | 15/15 | 45/45 | | |
| pneumoniae* | 038 | CFU/ml | | | | | | |
| | URV- | | | | | | | |
| | 036 | | | | | | | |
| | URV- | | | | | | | |
| | 022 | | | | | | | |
| | URV- | | | | | | | |
| | 031 | | | | | | | |
| | URV- | | | | | | | |
| | 155 | | | | | | | |
| Proteus | URV- | 25,000 | 15/15 | 15/15 | 15/15 | 45/45 | | |
| mirabilis* | 010 | CFU/ml | | | | | | |
| | URV- | | | | | | | |
| | 014 | | | | | | | |
| | URV- | | | | | | | |
| | 040 | | | | | | | |
| | URV- | | | | | | | |
| | 060 | | | | | | | |
| | URV- | | | | | | | |
| | 016 | | | | | | | |
| Staphylococcus | SJF- | 25,000 | 15/15 | 15/15 | 15/15 | 45/45 | | |
| aureus* | 038 | CFU/ml | | | | | | |
| | SJF- | | | | | | | |
| | 044 | | | | | | | |
| | SJF- | | | | | | | |
| | 034 | | | | | | | |
| | SJF- | | | | | | | |
| | 035 | | | | | | | |
| | SJF- | | | | | | | |
| | 041 | | | | | | | |
| Enterococcus | URV- | 100,000 | 3/3 | 3/3 | 3/3 | 9/9 | 45 | 21 |
| faecalis | 032-2 | CFU/mL | | | | | | |
| Escherichia | AR- | 100,000 | 3/3 | 3/3 | 3/3 | 9/9 | | |
| coli | 104 | CFU/mL | | | | | | |
| Klebsiella | AR- | 100,000 | 3/3 | 3/3 | 3/3 | 9/9 | | |
| pneumoniae | 557 | CFU/mL | | | | | | |
| Proteus | AR- | 100,000 | 3/3 | 3/3 | 3/3 | 9/9 | | |
| mirabilis | 156 | CFU/mL | | | | | | |
| Staphylococcus | SJF- | 100,000 | 3/3 | 3/3 | 3/3 | 9/9 | | |
| aureus | 035 | CFU/mL | | | | | | |
| Enterococcus | URV- | 15,000 | 3/3 | 3/3 | 3/3 | 9/9 | 45 | 21 |
| faecalis | 032-2 | CFU/mL | | | | | | |
| Escherichia | URV- | 15,000 | 3/3 | 3/3 | 3/3 | 9/9 | | |
| coli | 012 | CFU/mL | | | | | | |
| Klebsiella | URV- | 15,000 | 3/3 | 3/3 | 3/3 | 9/9 | | |
| pneumoniae | 155-1 | CFU/mL | | | | | | |
| Proteus | URV- | 15,000 | 3/3 | 3/3 | 3/3 | 9/9 | | |
| mirabilis | 023 | CFU/mL | | | | | | |
| Staphylococcus | ATCC- | 15,000 | 3/3 | 3/3 | 3/3 | 9/9 | | |
| aureus | 43300 | CFU/mL | | | | | | |
| Enterococcus | URV- | 20,000 | 3/3 | 3/3 | 3/3 | 9/9 | 45 | 21 |
| faecalis | 032-2 | CFU/mL | | | | | | |
| Escherichia | URV- | 20,000 | 3/3 | 3/3 | 3/3 | 9/9 | | |
| coli | 012 | CFU/mL | | | | | | |
| Klebsiella | URV- | 20,000 | 3/3 | 3/3 | 3/3 | 9/9 | | |
| pneumoniae | 155-1 | CFU/mL | | | | | | |
| Proteus | URV- | 20,000 | 3/3 | 3/3 | 3/3 | 9/9 | | |
| mirabilis | 023 | CFU/mL | | | | | | |
| Staphylococcus | ATCC- | 20,000 | 3/3 | 3/3 | 3/3 | 9/9 | | |
| aureus | 43300 | CFU/mL | | | | | | |
| Enterococcus | URV- | 25,000 | 3/3 | 3/3 | 3/3 | 9/9 | 45 | 21 |
| faecalis | 032-2 | CFU/mL | | | | | | |
| Escherichia | URV- | 25,000 | 3/3 | 3/3 | 3/3 | 9/9 | | |
| coli | 012 | CFU/mL | | | | | | |
| Klebsiella | URV- | 25,000 | 3/3 | 3/3 | 3/3 | 9/9 | | |
| pneumoniae | 155-1 | CFU/mL | | | | | | |
| Proteus | URV- | 25,000 | 3/3 | 3/3 | 3/3 | 9/9 | | |
| mirabilis | 023 | CFU/mL | | | | | | |
| Staphylococcus | ATCC- | 25,000 | 3/3 | 3/3 | 3/3 | 9/9 | | |
| aureus | 43300 | CFU/mL | | | | | | |
| Negative | — | — | 5/5 | 5/5 | 5/5 | 15/15 | 15 | 15 |
| Samples | | | | | | | | |

### Accuracy Verification

The accuracy verification study was intended to assess the precision of our assay for detecting uropathogens in urine clinical specimens in comparison to the current gold standard diagnostics (culture). The BIOTIA-ID Urine NGS Assay was tested using a combination of urine clinical specimens and contrived samples (whole organism microbial reference strains and clinical isolates spiked into negative clinical matrix). Deidentified clinically tested urine samples were obtained from a reference laboratory and stored accordingly until processing with BIOTIA-ID Urine NGS Assay. Urine clinical samples were collected based on the pathogens diagnosed by culture and were comprised of five of the most common urogenital pathogens (E. coli, E. faecalis, K. pneumoniae, P. mirabilis and S. aureus). At least 30 specimens per analyte were collected and when not available, contrived specimens were used (a total of 190 samples; 143 clinical specimens and 47 contrived). Contrived samples were used for S. aureus (n=30), P. mirabilis (n=9) and K. pneumoniae (n=8) and were processed with a spike in concentration (CFU/mL) close to the LoD (~2.5×-5×). This study was a randomized, double blinded study in which at least 30 positive specimens, clinical and/or contrived were tested per analyte. In addition, 20 specimens negative by culture were also tested.

Comparator testing was conducted to help address discrepancies between the gold standard (culture) and BIOTIA-ID Urine NGS Assay. We employed a combination of qPCR and Sanger sequencing to resolve such discrepancies. A detailed list of all assays used as a third comparator are described in Supplementary Table 8. We adjudicated a total of 104 discrepancies with qPCR of which 83.5% of discrepancies were determined to be true positives and 3.1% were true negatives. Similarly, 19 discrepancies were resolved by amplifying target sequences followed by bidirectional Sanger Sequencing. The forward and reverse sequences were analyzed for quality, trimmed, and aligned to generate a consensus sequence using Geneious Prime software (v. 2023.2.1). The consensus sequence was queried on NCBI Blast and checked against the NCBI Genbank database and NCBI RefSeq database. Results where the % identity is >97% and query cover is >99% are considered a match for the taxa identified on the databases. We adjudicated a total of 19 discrepancies of which 84.2% of discrepancies were determined to be true positives, with >99.25% identities. All comparator testing results are summarized in Supplementary Table 9.

The accuracy verification study was comprised of 190 total specimens (47 contrived, 143 urine clinical specimens) and after resolving discrepancies with a third comparator, yielded an overall of 97.35% sensitivity and 99.65% specificity (Table 9). Performance characteristics obtained for clinical specimens resulted in a 96.77% sensitivity and 99.58% specificity (Table 9).

Table 9 shows a summary of performance characteristics for the accuracy verification study.

TABLE 9
Table 9: Summary of Performance Characteristics
for Accuracy Verification Study

| Method | TP | TN | FN | FP | Total | Sensitivity | Specificity |
|---|---|---|---|---|---|---|---|
| Clinical | 210 | 8323 | 7 | 35 | 8575 | 96.77% | 99.58% |
| Contrived | 47 | 1598 | 0 | 14 | 1645 | 100.00% | 100.00% |
| Total | 257 | 9921 | 7 | 35 | 10220 | 97.35% | 99.65% |

### Conclusions—Bacterial Validation

The BIOTIA-ID Urine NGS Assay validation yielded an overall assay performance of 99.89% sensitivity and 99.94% specificity when evaluating the entire validation dataset (Table 10). Table 10 shows a detailed breakdown of the overall performance characteristics of this validation per key urogenital pathogen.

TABLE 10
Table 10: Summary of Performance Characteristics
of BIOTIA-ID Urine NGS Assay Validation Study

| Method | TP | TN | FN | FP | Total | Sensitivity | Specificity |
|---|---|---|---|---|---|---|---|
| Clinical | 210 | 8323 | 7 | 35 | 8575 | 96.77% | 99.58% |
| Contrived | 1099 | 43122 | 0 | 14 | 44048 | 100.00% | 99.97% |
| In Silico | 8119 | 272775 | 3 | 147 | 281044 | 99.96% | 99.95% |
| Total | 9431 | 332483 | 10 | 196 | 341933 | 99.89% | 99.94% |

### Fungal Addendum

BIOTIA-ID Urine NGS Assay has been extensively tested and validated for the detection of bacterial urogenital pathogens as described above. In this addendum, we sought to validate our comprehensive diagnostic test for the detection of bacterial and fungal urogenital pathogens from one sample, using the most prevalent fungal urogenital pathogens belonging to the Candida genus. This validation was designed to include a combination of urine clinical specimens and contrived spike-in samples using whole organism microbial reference strains and clinical isolates. The analytical validation was separated and conducted in four separate studies: specificity, sensitivity, reproducibility and accuracy. Across these studies, 528 samples were processed at the laboratory (35 clinical specimens, 493 contrived) and tested with BIOTIA-ID Urine NGS Assay. The BIOTIA-ID Urine NGS Assay validation yielded an overall assay performance of 99.98% sensitivity and 100% specificity when evaluating the entire fungal validation dataset (n=5098 analytes).

Whole fungal cells of key urogenital pathogens detected by our assay, genetically related organisms, and others commonly present in urine specimens were included in the specificity studies. A total of 13 fungal species and 37 total strains were evaluated in the laboratory. Whole organisms were spiked into negative urine matrix at a concentration of 25,000 CFU/mL and processed with the assay. The specificity study entailed testing contrived specimens which resulted in an overall sensitivity and specificity of 100%.

We also conducted an extensive in silico analysis to further evaluate the performance and specificity of the assay. In addition to the previously tested samples, 4,142 of simulated in silico samples were processed and tested for reportable fungal species. An overall sensitivity and specificity of 100% was obtained for simulated samples.

The sensitivity studies sought to determine the LoD of our assay in six fungal pathogens (Candida albicans, Candida auris, Candida glabrata, Candida krusei, Candida parapsilosis, and Candida tropicalis). Whole organisms were spiked into negative urine matrix at different CFU/mL concentrations and 6 total replicates were tested per concentration. A total of 214 contrived specimens were processed for this component of the validation. The LoD was determined as the lowest concentration in which 100% of the replicates (6/6) were detected. The overall LoD was <5,000 CFU/mL.

Finally, an inter/intra-assay reproducibility study was used to assess variability in the assay. Five different pools containing one clinical strain of Candida albicans, Candida auris, Candida glabrata, Candida krusei, Candida parapsilosis, and Candida tropicalis were tested. Each pool had different strains for each species (altogether 30 different strains) and were spiked into negative urine matrix at a concentration of 50,000 CFU/mL per analyte. A sixth pool containing one strain of Candida dubliniensis, Candida guilliermondii, Candida kefyr and Candida lusitaniae was also tested. Each pool was tested daily in three technical replicates for three consecutive days for a total of 15 replicates per pool, 45 replicates per analyte in addition to 27 negative samples. 99.4% of the analytes tested were reproducibly and correctly detected obtaining a sensitivity and specificity of 100% and 99.94%.

The accuracy verification study consisted of 156 total specimens (121 contrived, 35 urine clinical specimens). For this addendum, we were unable to collect 30 clinical specimens per analyte due to limited resources and time, therefore, contrived specimens spiked with two reference and/or clinical fungal isolates were used. To validate the presence of organisms in contrived specimens, one third of the specimens were spiked at 10-fold the LOD for each analyte or 10,000 CFU/mL. This study was a randomized, double blinded study. The clinical validation yielded an overall performance of 99.58% sensitivity and 100% specificity.

### Fungal Validation Sample Summary

Table 11 shows a summary of specimens processed in each study of the validation.

TABLE 11
Summary of Specimens Processed in Each Study of the Validation
Table 11

| Sample | | | | Accuracy | |
|---|---|---|---|---|---|
| Type | Specificity | Sensitivity | Reproducibility | Verification | Total |
| Clinical | — | — | — | 35 | 35 |
| Contrived | 41 | 214 | 117 | 121 | 493 |
| In Silico | 4142 | — | — | — | 4142 |
| NEC | 4 | 14 | 15 | 18 | 51 |
| NTC | 2 | 7 | 9 | 9 | 27 |
| PC | 4 | 9 | 9 | 9 | 31 |
| Total | 4193 | 244 | 150 | 192 | 4779 |

### Laboratory Specificity Experiments

Reference and clinical fungal isolates were tested and validated on the specificity component of the BIOTIA-ID Urine NGS Assay clinical validation. Whole organisms of key urogenital pathogens detected by the assay, genetically related organisms, and other commonly found organisms in urine specimens were tested by spiking whole fungal cells into negative urine matrix. A total of 13 fungal species and 36 strains were evaluated. Fungal isolates were spiked into negative urine matrix at a concentration of 25,000 CFU/mL and processed with the BIOTIA-ID Urine NGS Assay resulting in an overall sensitivity and specificity of 100%. A detailed summary of sensitivity and specificity for all fungal species tested in this addendum can be found in Table 12.

### In Silico Cross Reactivity

To analyze cross-reactivity in silico a total of 4,142 RefSeq genomes belonging to 244 fungal genera and 452 fungal species were used to simulate FASTQ files with 100,000 reads each. A random mutation rate of 0.5% was applied during simulation to introduce a reasonable degree of variability. Simulated read files were then run through BIOTIA-DX in an identical manner to sequenced laboratory samples and tested for key organisms. In this way, we could assess the potential for organisms outside our laboratory sample set to generate false positive results for key organisms. Overall, the sensitivity and specificity of in silico samples is 100%. This study didn't show crossreactive samples to any of the BIOTIA-ID Urine NGS Assay reportable taxa.

### Specificity Performance Characteristics

Table 12 shows a summary of performance characteristics of specificity study.

TABLE 12
Summary of Performance Characteristics of
Specificity Study (Contrived and In Silico)
Table 12

| Method | TP | TN | FN | FP | Total | Sensitivity | Specificity |
|---|---|---|---|---|---|---|---|
| Contrived | 41 | 410 | 0 | 0 | 451 | 100.00% | 100.00% |
| In silico | 4142 | 41420 | 0 | 0 | 45562 | 100.00% | 100.00% |
| Total | 4183 | 41830 | 0 | 0 | 46013 | 100.00% | 100.00% |

### Sensitivity

The LoD was determined by performing a 10-fold dilution series of whole organisms of interest (6 data points, 1E+01 to 1E+06 CFU/mL) and spiking these in triplicates into negative clinical urine matrix. Each LoD experiment was conducted in duplicates for 6 total replicates per concentration, per analyte tested. Each contrived sample tested was spiked with three different fungal species or three analytes tested per sample processed. After obtaining the lowest detectable concentration in the 10-fold dilution series experiments, a dilution series from 1E+01 to 5E+04 was used to fine-tune the LoD. A total of 214 contrived samples were tested in these experiments. The LoD was established when the target organism is detected in 95% of the samples which is equal to 6/6 replicates.

The LoD was assessed on six of the most prevalent fungal urogenital pathogens, (Candida albicans, Candida auris, Candida glabrata, Candida krusei, Candida parapsilosis, and Candida tropicalis) using fungal cells spiked into negative urine matrix. Fungal species were pooled in groups of three in these experiments to show LoD is not affected by multiple organisms in the sample. The LoD was reproducibly verified and determined based on a 100% positivity rate using the assay. The overall LoD was <5,000 CFU/mL. Specifically, the LoD determined for each species is as follows: 1,000 CFU/mL for C. glabrata, C. krusei, C. parapsilosis, C. tropicalis; 5,000 CFU/mL for C. albicans and C. auris (Table 13).

Table 13 shows a summary of results from the analytical sensitivity study per analyte.

TABLE 13
Table 13: Summary of Results from the Analytical Sensitivity Study per Analyte

| | Detection | CFU/mL | | | | | | | | | | | |
|---|---|---|---|---|---|---|---|---|---|---|---|---|---|
| | Limit | 2.5 × | 5 × | 7.5 × | 1 × | 5 × | 7.5 × | 1 × | 1.25 × | 2.5 × | 5 × | 1 × | 1 × |
| Analyte | (CFU/mL) | 10² | 10² | 10² | 10³ | 10³ | 10⁴ | 10⁴ | 10⁴ | 10⁴ | 10⁴ | 10⁵ | 10⁶ |
| Candida | 5,000 | 0/6 | 0/6 | 5/6 | 4/6 | 6/6 | 6/6 | 6/6 | 6/6 | 6/6 | 6/6 | 6/6 | 6/6 |
| albicans | | | | | | | | | | | | | |
| Candida | 5,000 | 0/6 | 0/6 | 0/6 | 4/6 | 6/6 | 6/6 | 6/6 | 6/6 | 6/6 | 6/6 | 6/6 | 6/6 |
| auris | | | | | | | | | | | | | |
| Candida | 1,000 | 0/6 | 0/6 | 0/6 | 6/6 | 6/6 | 6/6 | 6/6 | 6/6 | 6/6 | 6/6 | 6/6 | 6/6 |
| glabrata | | | | | | | | | | | | | |
| Candida | 1,000 | 0/6 | 0/6 | 0/6 | 6/6 | 6/6 | 6/6 | 6/6 | 6/6 | 6/6 | 6/6 | 6/6 | 6/6 |
| krusei | | | | | | | | | | | | | |
| Candida | 1,000 | 0/6 | 0/6 | 0/6 | 6/6 | 6/6 | 6/6 | 6/6 | 6/6 | 6/6 | 6/6 | 6/6 | 6/6 |
| parapsilosis | | | | | | | | | | | | | |
| Candida | 1,000 | 0/6 | 0/6 | 0/6 | 6/6 | 6/6 | 6/6 | 6/6 | 6/6 | 6/6 | 6/6 | 6/6s | 6/6 |
| tropicalis | | | | | | | | | | | | | |

### Inter/Intra-Reproducibility

Inter/intra assay reproducibility were combined into one experiment. Five different pools containing one clinical strain each of Candida albicans, Candida auris, Candida glabrata, Candida krusei, Candida parapsilosis, and Candida tropicalis were tested. Each pool had different strains for each species (altogether 30 different strains) and were spiked into negative urine matrix at a concentration of 50,000 CFU/mL per analyte. A sixth pool containing one strain of Candida dubliniensis, Candida guilliermondii, Candida kefyr and Candida lusitaniae was also tested. Each pool was tested daily in three technical replicates for three consecutive days for a total of 15 replicates per pool, 45 replicates per analyte for species in pools 1-5, 9 replicates of species in pool 6 and 27 negative samples. In summary, 100% of the analytes evaluated were reproducibly and correctly detected obtaining a sensitivity and specificity of 100%. (Table 14). Table 14 shows the number of samples in which the organism was detected over the number of samples tested in each pool (1-6) and day (1-3).

Table 14 shows a summary of inter/intra assay reproducibility results per analyte.

TABLE 14
Summary of Inter/Intra Assay Reproducibility Results per Analyte
Table 14

| | | | Day | Day | Day |
|---|---|---|---|---|---|
| Organism | Pool | Strain | One | Two | Three |
| Candida | 1 | 61093 | 3/3 | 3/3 | 3/3 |
| albicans | 2 | 10231 | 3/3 | 3/3 | 3/3 |
| | 3 | 14053 | 3/3 | 3/3 | 3/3 |
| | 4 | 90029 | 3/3 | 3/3 | 3/3 |
| | 5 | URV-018 | 3/3 | 3/3 | 3/3 |
| Candida | 1 | MYA-5000 | 3/3 | 3/3 | 3/3 |
| auris | 2 | MYA-5001 | 3/3 | 3/3 | 3/3 |
| | 3 | MYA-5002 | 3/3 | 3/3 | 3/3 |
| | 4 | MYA-5003 | 3/3 | 3/3 | 3/3 |
| | 5 | CBC B11903 | 3/3 | 3/3 | 3/3 |
| Candida | 1 | 22019 | 3/3 | 3/3 | 3/3 |
| parapsilosis | 2 | 90018 | 3/3 | 3/3 | 3/3 |
| | 3 | SJF-019 | 3/3 | 3/3 | 3/3 |
| | 4 | SJF-032 | 3/3 | 3/3 | 3/3 |
| | 5 | SJF-033 | 3/3 | 3/3 | 3/3 |
| Candida | 1 | 15126 | 3/3 | 3/3 | 3/3 |
| glabrata | 2 | 2001 | 3/3 | 3/3 | 3/3 |
| | 3 | 66032 | 3/3 | 3/3 | 3/3 |
| | 4 | 90030 | 3/3 | 3/3 | 3/3 |
| | 5 | MYA-2950 | 3/3 | 3/3 | 3/3 |
| Candida | 1 | 14243 | 3/3 | 3/3 | 3/3 |
| krusei | 2 | 34135 | 3/3 | 3/3 | 3/3 |
| | 3 | 90878 | 3/3 | 3/3 | 3/3 |
| | 4 | 6258 | 3/3 | 3/3 | 3/3 |
| | 5 | URV-019 | 3/3 | 3/3 | 3/3 |
| Candida | 1 | 9968 | 3/3 | 3/3 | 3/3 |
| tropicalis | 2 | 1369 | 3/3 | 3/3 | 3/3 |
| | 3 | 750 | 3/3 | 3/3 | 3/3 |
| | 4 | 13803 | 3/3 | 3/3 | 3/3 |
| | 5 | 66029 | 3/3 | 3/3 | 3/3 |
| Candida | 6 | 3949 | 3/3 | 3/3 | 3/3 |
| dubliniensis | | | | | |
| Candida | | 6260 | 3/3 | 3/3 | 3/3 |
| guillermondii | | | | | |
| Candida | | 66028 | 3/3 | 3/3 | 3/3 |
| kefyr | | | | | |
| Candida | | 34449 | 3/3 | 3/3 | 3/3 |
| lusitaniae | | | | | |

### Accuracy Verification

The accuracy verification study was intended to assess the precision of our assay for detecting uropathogens in urine clinical specimens in comparison to the current gold standard diagnostics (culture). The BIOTIA-ID Urine NGS Assay was tested using a combination of urine clinical specimens and contrived samples (whole organism microbial reference strains and clinical isolates spiked into negative clinical matrix). Deidentified clinically tested urine samples were obtained from a reference laboratory and stored accordingly until processing with BIOTIA-ID Urine NGS Assay. This study was a randomized, double blinded study in which at least 30 positive specimens, clinical and/or contrived were evaluated per analyte. In addition, 15 specimens negative by culture were also tested. For this addendum, we were unable to collect 30 clinical specimens per analyte due to limited resources and time, therefore, contrived specimens spiked with two reference and/or clinical fungal isolates were used. Since majority of specimens were contrived, one third of the specimens were spiked at 10-fold the LOD for each analyte or 10,000 CFU/mL.

Comparator testing was conducted to help address discrepancies between the gold standard (culture) and BIOTIA-ID Urine NGS Assay. We amplified the fungal ITS2 and performed bidirectional Sanger sequencing to resolve such. The ITS2 forward and reverse sequences were analyzed for quality, trimmed, and aligned to generate a consensus sequence. The consensus sequence was queried on NCBI Blast and checked against the Genbank database and RefSeq. Samples where the % identity is >97% are considered a match for the taxa identified on the databases. We adjudicated a total of 21 discrepancies of which 100% of discrepancies were determined to be true positives.

The accuracy verification study consisted of 156 total specimens (121 contrived, 35 urine clinical specimens) and after resolving discrepancies with a third comparator, yielded an overall of 99.58% sensitivity and 100% specificity. Performance characteristics obtained for clinical specimens resulted in a 100% sensitivity and 100% specificity and for contrived specimens 99.50% sensitivity and 100% specificity (Table 15).

Table 15 shows a summary of performance characteristics for the accuracy verification study.

TABLE 15
Table 15: Summary of Performance Characteristics
for the Accuracy Verification Study

| Method | TP | TN | FN | FP | Total | Sensitivity | Specificity |
|---|---|---|---|---|---|---|---|
| Clinical | 36 | 530 | 0 | 0 | 560 | 100.00% | 100.00% |
| Contrived | 199 | 2000 | 1 | 0 | 2200 | 99.50% | 100.00% |
| Total | 235 | 2530 | 1 | 0 | 2760 | 99.58% | 100.00% |

### Conclusions—Fungal Validation

The BIOTIA-ID Urine NGS Assay validation yielded an overall assay performance of 99.98% sensitivity and 100% specificity when evaluating the entire fungal validation dataset. Table 16 shows a detailed breakdown of the overall performance characteristics of this validation per key urogenital pathogen.

Table 16 shows performance characteristics of BIOTIA-ID urine NGS assay fungal validation study

TABLE 16
Table 16: Performance Characteristics of BIOTIA-ID
Urine NGS Assay Fungal Validation Study

| Method | TP | TN | FN | FP | Total | Sensitivity | Specificity |
|---|---|---|---|---|---|---|---|
| Clinical | 30 | 530 | 0 | 0 | 560 | 100.00% | 100.00% |
| Contrived | 925 | 9240 | 1 | 2 | 10168 | 99.89% | 99.98% |
| In Silico | 4142 | 41420 | 0 | 0 | 45562 | 100.00% | 100.00% |
| Total | 5097 | 51190 | 1 | 2 | 56290 | 99.98% | 100.00% |

### Example 2: Identification of Antibiotic Resistance Markers

This example details a successful implementation of a urine NGS assay described herein. In this example the BIOTIA-DX pipeline, was implemented for detecting beta-lactam, sulfonamide, trimethoprim, and vancomycin resistance markers, which are relevant in UTIs. The BIOTIA-DX tool used the machine learning systems described above to classify novel or rare mutations more effectively and compares novel genes to a global database of sequences. The analysis identified prominent antimicrobial resistance genes related to UTI/STI from sequencing data and can map AMR to a particular pathogen with high accuracy. The assay was implemented as follows:

### Method for Detecting Antimicrobial Resistance Genes:

1) Samples were collected. See FIG. 1, module 1. De-identified, left-over urine specimens are collected were processed under IRB approval;
2) Midstream clean-catch urine specimens were preserved in Urine Transport Tubes (UTT);
3) Genomic DNA was isolated from clinical specimens using a QIAcube-MDx extraction and quantified with Qubit-Flex. See FIG. 1, module 2.

### Library Preparation and Sequencing See FIG. 1, Module 3:

1) Metagenomic libraries were prepared using the Illumina DNA Prep Library preparation kit;
2) Libraries are quality checked for size and concentration using Tapestation 4200 and Qubit-Flex, respectively;
3) Libraries were pooled in 24-plex reactions and sequenced on an Illumina NextSeq 550 platform using a NextSeq 500/550 Mid Output kit set to 150 bp single-end reads with i5 and i7 indexes. See FIG. 1, module 4:

### Data Processing and Analysis:

1) Samples were processed with the BIOTIA-DX pipeline. Read preprocessing includes the removal of low-quality reads and human reads;
2) Clean reads were pseudo-aligned to a large database of microbial genomes in a coarse classification step;
3) Organisms identified from coarse classification were filtered for identification quality and sent to a fine classification step;
4) Reads were realigned to curated pangenomes for each organism, and summary statistics are generated.
5) The BIOTIA-DX pipeline incorporating the pangenomic index and the duplication index.
   These statistics were fed into a machine learning classifier, which assigns a confidence score for whether the organism is present or absent based on a duplication index and a pangenomic index. See FIG. 1, module 5:

### AMR Gene Identification:

1) Preprocessed reads were assembled with the Megahit de novo assembler tool, and contigs are screened with NCBI AMRFinder;
2) In parallel, preprocessed reads are realigned to the BIOTIA-DX AMR database to identify markers on a higher level. For gene-level prediction, k-mer and variant calling-based methods are combined.

### 2. Results:

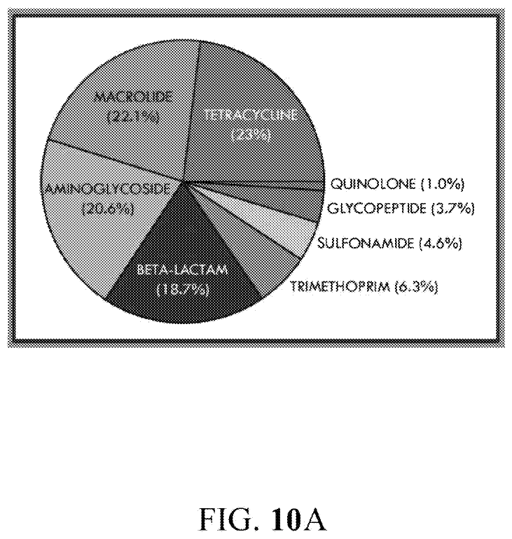

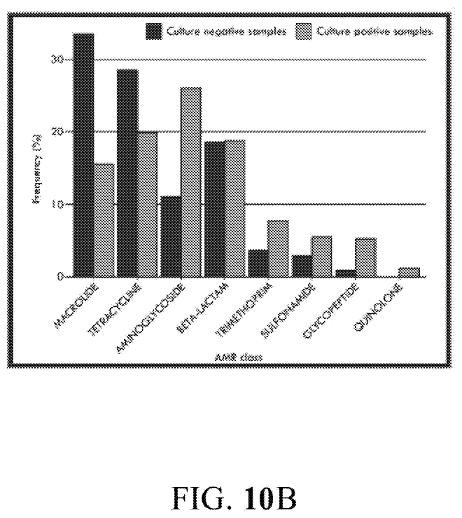

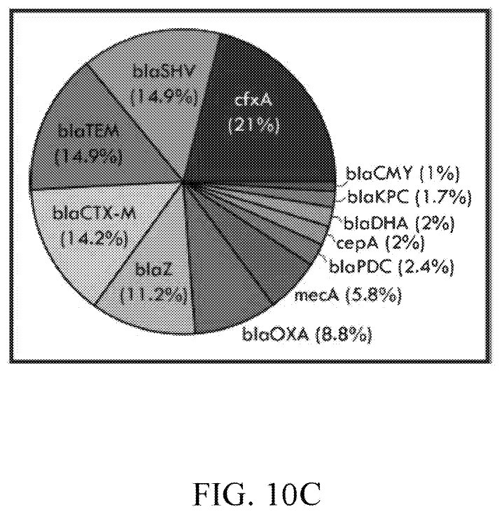

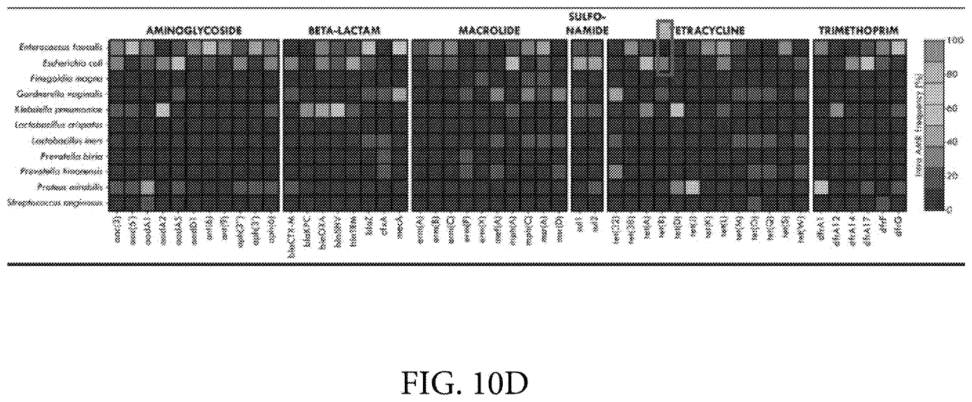

The BIOTIA-DX tool demonstrates high sensitivity (86-100%) and specificity (98-100%) in detecting AMR genes. FIG. 10A is a chart demonstrating the distribution of AMR classes in sample cohort. Tetracycline (23%), macrolide (22.1%), aminoglycoside (20.6%), beta-lactam (18.7%) proved to be the most abundant in the sample cohort with a near equal frequency. See FIG. 10A. Trimethoprim (6.3), sulfonamide (4.6%), glycopeptide (3.7%), and quinolone (1.0%) resistance markers were also identified. FIG. 10B quantitates the results of AMR profiles in cultures positive vs culture negative samples. Briefly, beta-lactam markers represented a balanced frequency (~18%) in both cultures negative and positive specimen. See FIG. 10B. FIG. 10C further specifies detected beta-lactam resistance markers. The cfxA class-A betalactamase was detected with highest frequency, followed by blaCTX-M, blaSHV and blaTEM extended spectrum beta-lactamases. See FIG. 10C.

Notably, certain bacteria harbored specific antimicrobial resistance gene (ARG) marker profiles. See FIG. 10D. Based on the intra-ARG frequency of the samples contained with the most prevalent species, some ARGs showed >90% abundance in samples contained the following species:
Enterococcus faecalis, aminoglycoside: aadD1, ant(9); Tetracycline: tet(38), tet(K), tet(S);
Eschericia coli: Trimethoprim: dfrA14, dfrA17
Klebsiella pneumoniae: Beta-lactam: blaKPC; Trimethoprim: dfrA12. See FIG. 10D.

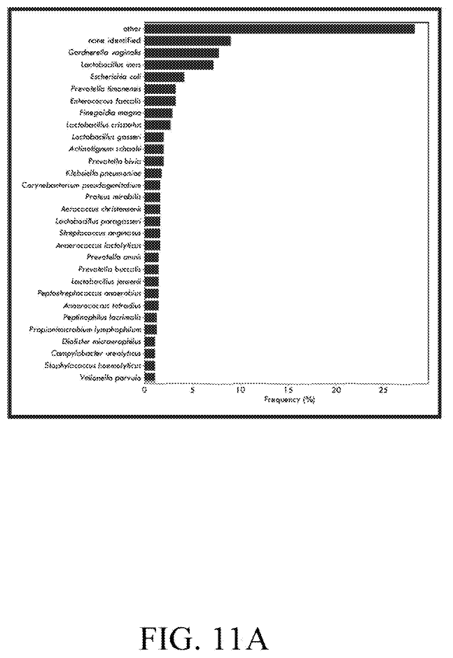

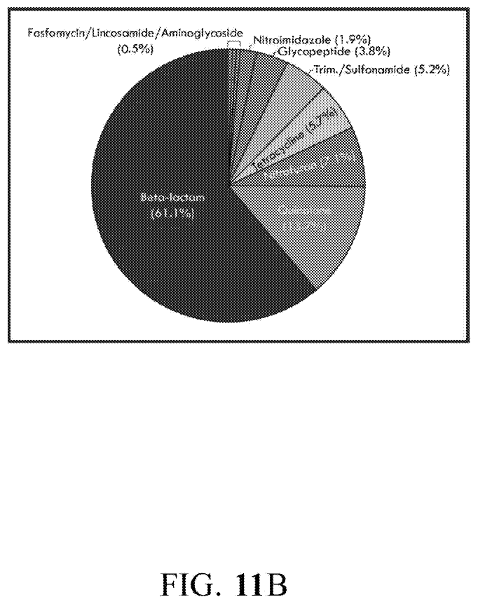

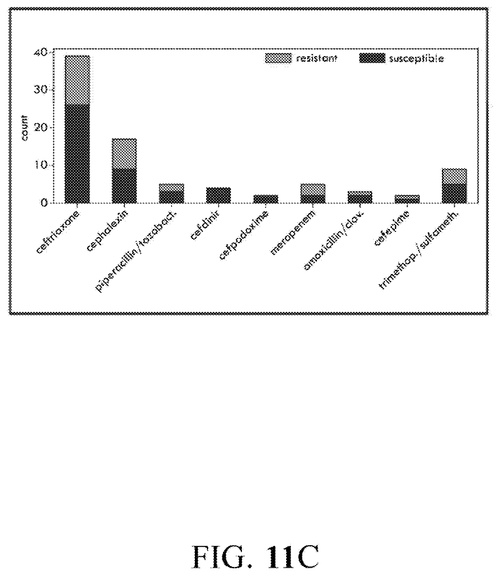

Furthermore, retrospective analysis of culture-negative specimens identified key urogenital pathogens, the underlying taxonomic composition, and resistance gene markers (FIGS. 11A-C). FIG. 11A illustrates the detected species in culture-negative specimens. Overall in 26% of the cases, the identified pathogens that were missed by standard of care (culture) had resistance against the empirically prescribed antimicrobials that may lead to therapeutic failure. In 40-45% of the cases where, ceftriaxone or cephalexin therapy were initiated beta-lactam resistance was detected. NGS has the potential to achieve adequate diagnosis and guide targeted therapy-especially in cases with complicated and recurrent UTI. FIG. 11B illustrates antibiotics prescribed for UTI treatment. FIG. 11C illustrates resistance numbers against the prescribed antibiotics.

In sum, a distinct ARG distribution was observed depending on the presence of different pathogens. An assay of the disclosure identified AMR with high accuracy, mapped AMR to a particular pathogen, and detected potentially novel markers that arise/arose through mutation.

### Conclusion

The BIOTIA-DX tool can identify pathogens and resistance genes missed by standard culture methods, which may lead to therapeutic failure. In cases where ceftriaxone or cephalexin therapy was initiated, beta-lactam resistance was detected, highlighting the tool's potential to guide targeted therapy, especially in complicated and recurrent UTI cases.

### Example 3: Detection of Bacterial Vaginosis Associated Organisms in Urine

This example details a successful implementation of a urine NGS assay described herein. Bacterial vaginosis (BV) is the most common cause of vaginal dysbiosis in women, affecting approximately 21.2 million women aged 14-49 in the United States. Traditional methods for diagnosing BV typically involve vaginal swabs and a combination of microscopy and PCR tests. However, these methods have limitations, including invasiveness and the potential for inaccurate results due to sample contamination or improper collection.

### Method for Detecting Bacterial Vaginosis:

1) Sample Collection: Midstream clean-catch urine specimens are collected and preserved in Urine Transport Tubes (UTT);
2) DNA Extraction: Genomic DNA is isolated from the urine specimens using a QIAcube-MDx extraction system;
3) Library Preparation: Metagenomic libraries are prepared using the Illumina DNA Prep Library preparation kit;
4) Sequencing: The libraries are sequenced on an Illumina NextSeq 550 platform using a NextSeq 500/550 Mid Output kit set to 150 bp single-end reads with i5 and i7 indexes;
5) Clean reads were pseudo-aligned to a large database of microbial genomes in a coarse classification step;
6) Organisms identified from coarse classification were filtered for identification quality and sent to a fine classification step;
7) Reads were realigned to curated pangenomes for each organism, and summary statistics are generated.
8) The BIOTIA-DX pipeline incorporating the pangenomic index and the duplication index.

These statistics are fed into a machine learning classifier which assigns a confidence score for the presence or absence of the organism. The NGS-Nugent Score was provided: The microbial profiles of the urine specimens were clustered, and a Nugent score was developed based on the presence and relative load of BV-associated bacteria and commensal Lactobacillus species.

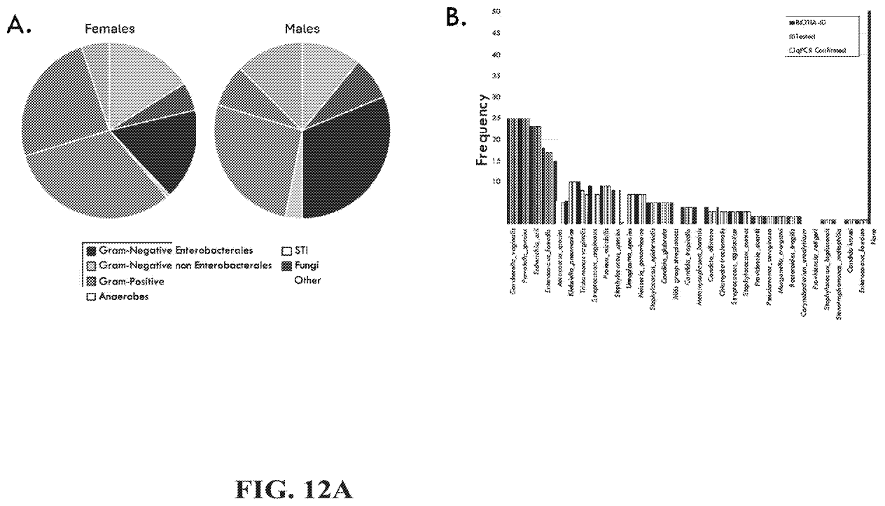

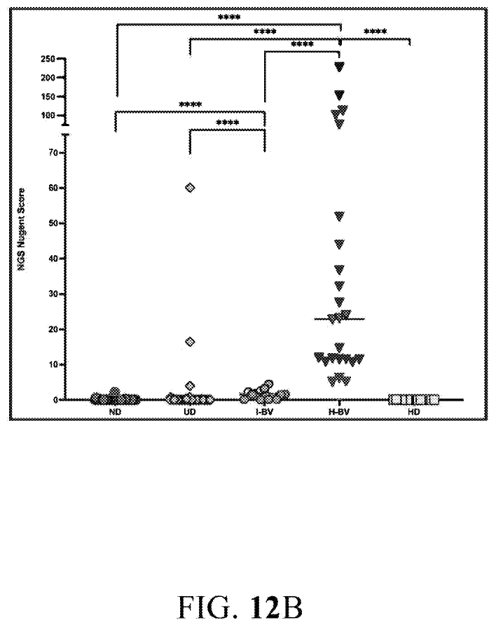

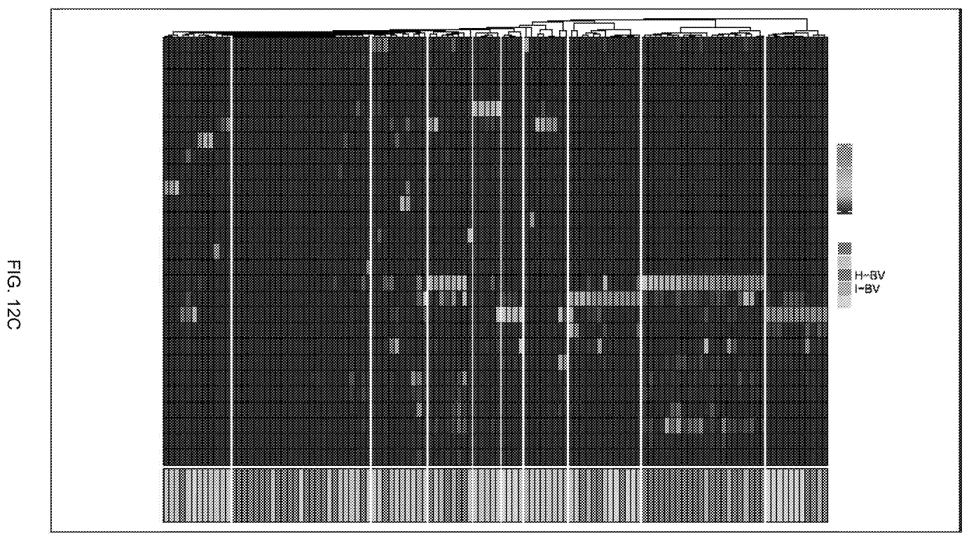

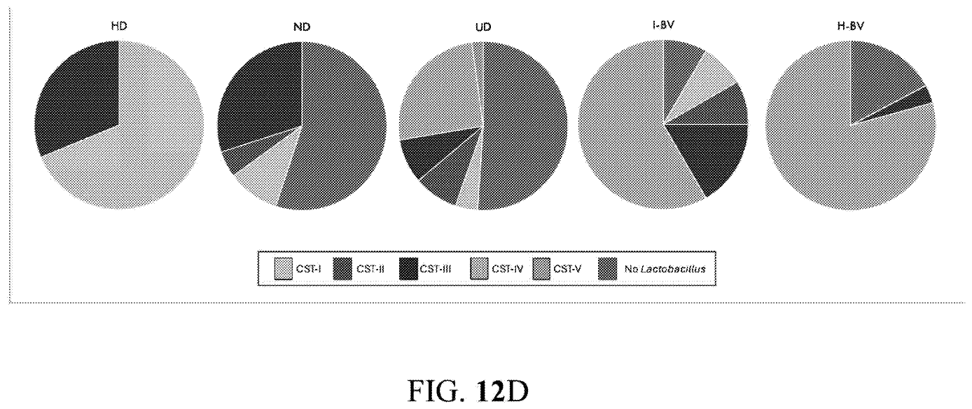

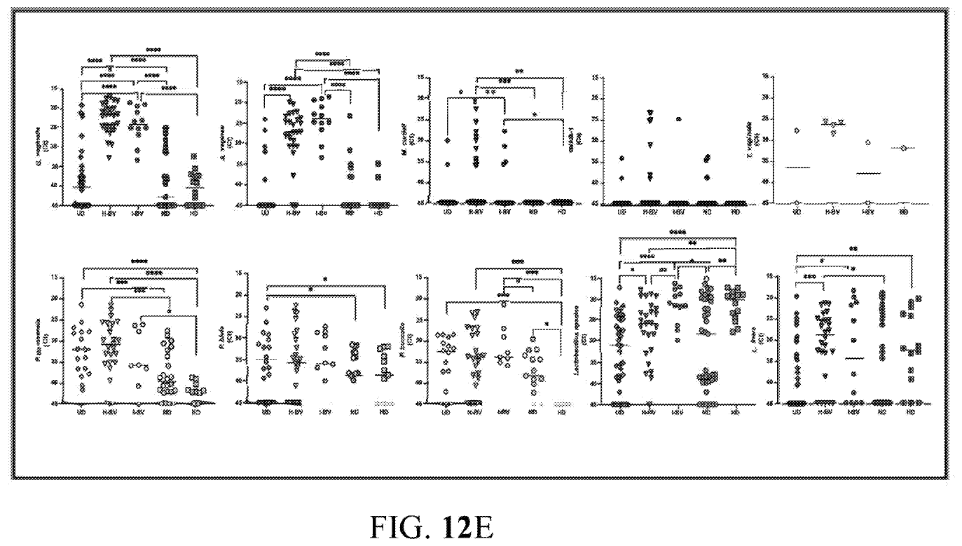

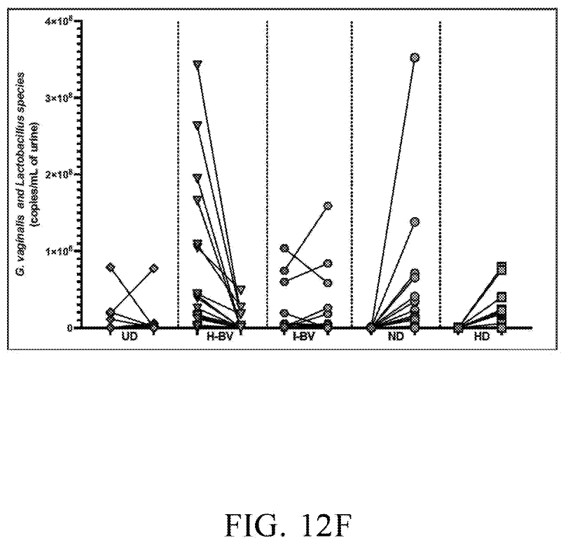

FIGS. 12A-F (FIG. 12A-F) are charts depicting an application of the urine assay of the disclosure in detecting bacterial vaginosis associated organism in urine. FIG. 12A describes that an assay of the disclosure, the BIOTIA-ID detected urogenital pathogens in 62% of the symptomatic CN urine specimens, while HD urine specimens did not have urogenital pathogens at infection levels. FIG. 12A(a) is a pie chart illustrating the distribution of microbial taxa groups in male and female samples. 31% of uropathogens detected in males belong to Gram Negative Enterobacterales, followed by 27% Gram Positive taxa, while females had 32% Gram positive and 25% belonging to other taxa (Gardnerella vaginalis). FIG. 12A(b) is a chart showing distribution of microbial species detected with BIOTIA-ID Urine NGS Assay. Most of the taxa detected by NGS was validated with a qPCR comparator to confirm presence and levels of organisms with a comparator method. (*) Indicates STI taxa that are not reported with BIOTIA-ID but are relevant to this study.

FIG. 12B is a chart describing the NGS Nugent Score provided by an assay of the disclosure. Briefly, FIG. 12B shows the stratification of female urine specimens based on the BIOTIA-ID result. The NGS microbial profiles and diversity, the presence of BV associated bacteria and/or commensal Lactobacillus species and the total bacterial load was used to develop an NGS-Nugent score. ND are specimens with no uropathogen, or BV-associated bacteria detected by NGS, and UD includes specimens with at least one uropathogen detected. H-BV specimens showed high diversity, high load and high NGS-Nugent score (5-200), while I-BV showed moderate levels of BV-associated bacteria with low levels of Lactobacillus species. (*, p<0.05; **, p<0.01; ***, p<0.001; Mann-Whitney U test).

FIG. 12C is a heat map depicting microbial profiles of CN (n=118) and HD (n=17) female urine specimens tested with BIOTIA-ID Urine NGS Assay. (A) Heatmap shows the distribution of uropathogens, BVassociated bacteria and commensal Lactobacillus species found in the tested urine specimens. Hierarchical clustering of microbial taxa detected by NGS showed a distinct group predominated by specimens with high diversity and abundance of BV-associated organisms while most of the HD specimens are clustering with ND specimens based on the Lactobacillus profiles. UD specimens are separating based on the detected uropathogen and whether they had detectable levels of BV-associated organisms and/or Lactobacillus species.

FIG. 12D are pie charts depicting community state types (CST's) were used to characterize the microbial dynamics observed in each NGS-Nugent Score. Relative abundances of the predominant lactic acid bacteria in healthy vagina were used to type the bacteria community groups, known as community state types (CSTs) (Ravel, J., et al. 2011). The CSTs are grouped as CST I, II, III, IV, V, with each of the CSTs dominated by L. crispatus, L. gasseri, L. iners, polymicrobial flora including Lactobacillus and bacterial vaginosis-associated bacteria (BVAB), and L. jensenii, respectively. CST-IV was most predominant in H-BV (79% of samples) and I-BV (58% of samples), while CST-I and CST-III were more frequent in HD and ND group. Additionally, over half of both ND and UD had no detectable levels of vaginal Lactobacillus species.

FIG. 12E are charts depicting the relative semi quantification of 7 BV-associated bacteria, Lactobacillus iners, Lactobacillus spp, and Trichomonas vaginalis was performed on female urine specimens. Specimens are grouped based on their NGS-Nugent Score and significant statistical differences are shown. Overall, higher relative levels of BV-associated bacteria were detected on H-BV and I-BV samples when compared to UD, ND and HD samples (*, p<0.05; **, p<0.01; ***, p<0.001; Mann-Whitney U test).

FIG. 12F is a chart demonstrating that quantification of G. vaginalis and Lactobacillus species supports the BV-stratification performed with the developed NGSNugent score. H-BV specimens display high levels of G. vaginalis and depleted levels of Lactobacillus species, while ND and HD specimens resulted in the inverse relationship of these two species. Standard curves were not generated for all organisms; thus, we relied on relative semi quantification to see trends in other bacterial taxa as shown above.

### CONCLUSION

The method was validated using a cohort of symptomatic and healthy donor urine specimens. The BIOTIA-ID Urine NGS Assay demonstrated high accuracy with 97% sensitivity and 99% specificity for detecting BV-associated organisms. The assay was able to identify Gardnerella vaginalis, Prevotella species, and other BV-associated bacteria with high confidence, providing a reliable non-invasive diagnostic tool for BV.

The invention provides a novel, non-invasive method for diagnosing bacterial vaginosis using next-generation sequencing technology. The method offers high sensitivity and specificity, making it a valuable tool for clinical diagnostics and improving patient outcomes by enabling timely and precise diagnosis of BV.

While certain embodiments have been described, these embodiments have been presented by way of example only and are not intended to limit the scope of the present disclosures. Indeed, the novel methods, apparatuses, modules, instruments and systems described herein can be embodied in a variety of other forms; furthermore, various omissions, substitutions and changes in the form of the methods, apparatuses, modules, instruments and systems described herein can be made without departing from the spirit of the present disclosures. The accompanying claims and their equivalents are intended to cover such forms or modifications as would fall within the scope and spirit of the present disclosures.

What is claimed is:

1. A urine assay, comprising the steps of:
extracting a set of genomic nucleic acids from a urine sample of a subject, whereby the extracting comprises the steps of:
removing one or more inhibitors from the urine sample;
submitting one or more microorganisms in the urine sample to one or more cycles of heating and cooling thus releasing a subset of genomic nucleic acids from the urine sample;
converting the subset of genomic nucleic acids into a library of fragments that can be sequenced on a next-generation sequencing instrument;
sequencing the extracted genomic DNA in the next-generation metagenomics sequencing instrument thereby generating a plurality of sequencing reads;
uploading the plurality of sequencing reads into a system comprising:
a memory comprising computer readable instructions stored thereon; and
a processor configured to execute the computer readable instructions to:
receive the plurality of sequencing reads;
determine whether the plurality of sequencing reads meet one or more thresholds in a pangenome index or a duplication index; and
outputting a result of the assay.

2. The urine assay of claim 1, wherein the result of the assay is an identification of one or more antibiotic resistance markers in the sample.

3. The urine assay of claim 1, wherein the result of the assay is an identification of bacterial vaginosis.

4. The urine assay of claim 3, further comprising providing a Nugent score in the identification of bacterial vaginosis.

5. The urine assay of claim 1, the pangenome index integrates sequences from the plurality of sequencing reads into a unified structure, and calculates its index as the number of genomes in which the plurality of sequencing reads are found, divided by the total number of genomes.

6. The urine assay of claim 1, wherein the duplication index is calculated as the number of pangenomes in which gene duplications are found, divided by the total number of pangenomes in the taxonomic group and expressed as a percentage.

7. The urine assay of claim 1, wherein the one or more thresholds comprise the pangenome index and the duplication index.

8. The urine assay of claim 7, wherein the incorporation of the pangenome index increases a sensitivity of the assay to greater than 85% percent.

9. The urine assay of claim 7, wherein the incorporation of the duplication index increases a specificity of the assay to greater than 85% percent.

10. The urine assay of claim 1, wherein the pangenome index and the duplication index identify greater than 70 distinct pangenomes associated with at least 70 distinct microorganisms.

11. The urine assay of claim 1, wherein the assay simultaneously screens the urine sample for at least 78 distinct microorganisms.

12. The urine assay of claim 1, wherein the one or more thresholds includes a tertiary fold index.

13. The urine assay of claim 12, wherein the tertiary fold index is a prediction of a tertiary fold from a set of sequencing reads in the plurality of sequencing reads.

14. A system for organizing and representing genetic diversity, comprising:
a memory comprising computer readable instructions stored thereon; and
a processor configured to execute the computer readable instructions to:
receive a genome sequence from a reference database;
determine whether the genome sequence meets a threshold;
if the genome sequence does not meet the threshold, select another genome sequence from the reference database;
if the genome sequence meets the threshold, process the genome sequence according to a pangenome index and a duplication index,
wherein the pangenome index integrates sequences from diverse individuals into a unified structure, facilitating rapid searches and analysis of genetic variations, and the duplication index quantifies gene duplications, offering insights into evolutionary and functional consequences;
generate a score for the processed genome sequence based on input from a human expert, wherein the score comprises metrics, percentages, thresholds, or ranges;
transmit the score to the reference database for storage, contributing to the refinement of the database; and
continuously update and refine the threshold based on classifications generated in the reference database, wherein the continuous updating is provided by the human expert via a device such that the threshold is updated in real-time.

15. The system of claim 14, wherein the genetic variations include single nucleotide polymorphisms, insertions, deletions, and structural variations.

16. The system of claim 14, wherein the duplication index reflects the frequency and distribution of duplicated sequences.

17. The system of claim 14, wherein the processor is further configured to execute the computer readable instructions to:
generate a coarse classification database using thresholds determined by the human expert to filter results, wherein the coarse classification database organizes data into broader, more general categories compared to fine-grained classifications, grouping similar items or concepts into higher-level classes, simplifying complex datasets, and reducing dimensionality;
iteratively refine the thresholds based on data;
build a fine classification database using the coarse classification database, wherein the fine classification database organizes biological data into highly specific and detailed categories, allowing for precise differentiation between closely related entities such as genes, proteins, or organisms, enabling researchers to identify subtle differences and make granular distinctions;
determine a set of metrics by the human expert to evaluate an alignment, wherein some individual metrics are more novel than others;
iteratively refine the choice of metrics based on data; and
generate the score using the machine learning algorithm trained on the data with known classifications.

18. The system of claim 17, wherein the score is used to update a results summary section of a report, and the report is structured to include both human-readable formats and machine-readable formats accessible via an Application Programming Interface (API).

19. A computer-implemented method for organizing and representing genetic diversity, comprising:
receiving a genome sequence from a reference database;
determining whether the genome sequence meets a threshold;
if the genome sequence does not meet the threshold, selecting another genome sequence from the reference database;
if the genome sequence meets the threshold, processing the genome sequence with novel metrics, including a pangenome index and a duplication index, wherein the pangenome index integrates sequences from diverse individuals into a unified structure, facilitating rapid searches and analysis of genetic variations, and the duplication index quantifies gene duplications, offering insights into evolutionary and functional consequences;
generating a score for the processed genome sequence based on input from a human expert, wherein the score comprises metrics, percentages, thresholds, or ranges;
transmitting the score to the reference database for storage, contributing to the refinement of the database; and
continuously updating and refining the threshold based on classifications generated in the reference database, wherein the continuous updating is provided by the human expert via a device such that the threshold is updated in real-time.

20. The method of claim 19, wherein the genetic variations include single nucleotide polymorphisms, insertions, deletions, and structural variations.

21. The method of claim 19, wherein the duplication index reflects the frequency and distribution of duplicated sequences.

22. The method of claim 19, further comprising:
generating a coarse classification database using thresholds determined by the human expert to filter results, wherein the coarse classification database organizes data into broader, more general categories compared to fine-grained classifications, grouping similar items or concepts into higher-level classes, simplifying complex datasets, and reducing dimensionality;
iteratively refining the thresholds based on data;
building a fine classification database using the coarse classification database, wherein the fine classification database organizes biological data into highly specific and detailed categories, allowing for precise differentiation between closely related entities such as genes, proteins, or organisms, enabling researchers to identify subtle differences and make granular distinctions;
determining a set of metrics by the human expert to evaluate an alignment, wherein some individual metrics are more novel than others;
iteratively refining the choice of metrics based on data; and
generating the score using the machine learning algorithm trained on the data with known classifications.

23. The method of claim 22, wherein the score is used to update a results summary section of a report, and the report is structured to include both human-readable formats and machine-readable formats accessible via an Application Programming Interface (API).
